(19) United States
(12) Patent Application Publication
(10) Pub. No.: US 2008/0233158 A1
(43) Pub. Date: Sep. 25, 2008

(54) Two-coat makeup product, its uses, and makeup kit comprising the product

(75) Inventors: Xavier Blin, Paris (FR); Caroline Lebre, Thiais (FR); Sophie Salem, Villejuif (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Ore al S.A.

(21) Appl. No.: 12/003,382

(22) Filed: Dec. 21, 2007

Related U.S. Application Data
(63) Continuation of application No. 10/784,959, filed on Feb. 25, 2004, now abandoned.

(30) Foreign Application Priority Data
Feb. 25, 2003 (FR) 03 50036

(51) Int. Cl. A61K 8/02; A61K 31/74; A61P 43/00

(52) U.S. Cl. 424/401; 424/78.03

(57) ABSTRACT

A cosmetic makeup product comprising at least two compositions, the first composition comprising a physiologically acceptable medium and the second composition comprising a mixture of at least one high molecular weight polymer having a weight-average molecular mass of greater than or equal to 200,000 g/mol and at least one low molecular weight non-volatile liquid silicone compound having a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 to 120 Pa·s; a makeup method and a makeup kit comprising the cosmetic makeup product. The cosmetic makeup product may, for example, be a lipstick.

This application claims benefit of U.S. Provisional Application No. 60/452,107, filed Mar. 10, 2003.

Disclosed herein is a new cosmetic makeup product comprising at least two compositions which can be applied in succession to the skin of both the face and the human body, to the upper and lower eyelids of human beings, to the lips and to the epidermal derivatives, such as the nails, eyebrows, eyelashes and hair, and also to a two-coat makeup method for the face and human body.

At least one of the compositions may be provided in a form chosen from loose and compact powders, foundations, blushes, eyeshadows, concealers, lipsticks, lip balms, lipglosses, lip pencils, eye pencils, mascaras, eyeliners, nail varnishes, and body makeup and skin coloring products.

The cosmetic makeup product may provide a glossy, taste-free, and/or comfortable makeup which may have good coverage and staying power, which may not transfer and which may go on easily.

Disclosed herein is thus a composition for caring for and/or making up a keratin material such as the skin of both the face and the human body, including the scalp, the epidermal integuments, such as the eyelashes, eyebrows, nails and hair, and also the lips and upper or lower eyelids of human beings.

“Non-transfer” makeup compositions for the lips and skin may have the advantage of forming a deposit which at least in part is not deposited on the substrates with which they are brought into contact such as, glasses, clothes, and cigarettes. Known non-transfer compositions, for example, the compositions described in U.S. Pat. No. 6,074,654 and Patent Application No. WO 02/067877, are generally based on silicone resins and volatile silicone oils and, although they provide improved staying properties, they can have the drawback of leaving on the skin and lips, following evaporation of the volatile silicone oils, a film which over time may become uncomfortable (giving sensations of drying and tightening), thereby causing a certain number of women not to use this type of lipstick.

To remedy at least some of these disadvantages, the present inventors contemplated the production of makeup compositions comprising particles of polymer dispersed and surface-stabilized by a stabilizer in a liquid fatty phase, as described in Patent No. EP-A-0 930 060. These compositions may not provide the kind of truly glossy makeup often sought after by consumers; thus, the present inventors further proposed applying to this composition a topcoat comprising a mixture of a silicone oil with a viscosity of 20 cSt and a silicone oil with a viscosity of 10,000 cSt (see Patent Application No. EP 1 249 223). That composition, however, may have the disadvantage of migrating and of becoming oily.

The company Kose, moreover, in Patent Application No. JP-A-0 5 221 829, proposed the use of a gel based on perfluorinated substances which is applied over a lipstick film so as to prevent its transfer to other surfaces, the gel being incompatible with the lipstick film.

Although the use of perfluorinated oils does ensure incompatibility between the gel and the lipstick film, and thus provide properties of staying power and absence of transfer, these formulations may have the disadvantage of providing poor cosmetic properties, because the lipstick film may become oily and may also migrate, which is unacceptable to consumers.

In Patent Application No. WO-A-97/17057, the company Procter & Gamble describes a method of enhancing staying and non-transfer properties, comprising applying two compositions one over the other. These two compositions have the following physicochemical criteria:
overall Hildebrand solubility parameters less than 8.5 (cal/cm³)^{1/2}, for the composition applied first and
the presence of oil whose calculated partition coefficient ClogP is at least 13, for the topcoat.

However, these compositions, given by way of example, may provide an uncomfortable makeup.

Patent Application No. WO 02/067877 describes a method of improving the aesthetic properties of a non-transfer composition, comprising applying a second composition to the film of the non-transfer composition. Some of the products described in WO 02/067877 may have an unpleasant odor and may be sticky; while other products may lack sufficient gloss and transparency.

Finally, U.S. Pat. No. 6,001,374 to Nichols proposes a multi-layer makeup system which involves using a base composition, comprising a resin which is soluble in alcohol and insoluble in water, and a topcoat composition, which comprises a silicone compound. The silicone compounds cited as examples, for example, the polydimethylsiloxane with a viscosity of 20,000,000 cSt and a molecular weight of 500,000 g/mol, in solution in a polydimethylsiloxane with a viscosity of 5 cSt and a molecular weight of 800 g/mol, in proportions by mass of 15/85 (reference product SF 1236), may be excessively sticky.

At least one aim of the present inventors was to provide a makeup product comprising at least two compositions to be applied one over the other, also referred to as a two-coat makeup product.

Thus, disclosed herein is a makeup product which can have at least one of the following advantages: not transferring, not migrating, staying power, comfort, absence of drying, gloss, absence of stickiness, and ease of spreading. These advantages have not heretofore been satisfactorily obtained.

The present inventors have found surprisingly that the combination of a first composition comprising a physiologically acceptable medium and a second composition comprising a mixture of at least one high molecular weight polymer having a weight-average molecular mass of greater than or equal to 200 000 g/mol and at least one low molecular weight non-volatile liquid silicone compound with a weight-average molecular mass of less than 200 000 g/mol, wherein the at least one high molecular weight polymer and at least one low molecular weight non-volatile liquid silicone compound are present in proportions such that the mixture has a specific viscosity, may provide a glossy two-coat makeup which may not migrate, not transfer, and/or have no taste, while going on smoothly and being comfortable over time (i.e., no drying, no tightening).

It has also been found that the second composition may, for example, have at least one of the following advantageous qualities: ease of spreading, adhesion, and a creamy and pleasant feel.

These properties of staying power and absence of taste, transfer and migration, in conjunction with a glossy appearance and comfort, make the two-coat makeup product disclosed herein a product which may, for example, be suitable for producing lip makeup products, such as lipsticks and lipglosses or eye makeup products such as mascaras, eyeliners and eyeshadows.

As used herein, the term “liquid fatty phase” means any non-aqueous medium which is liquid at ambient temperature. (25° C.) and atmospheric pressure (760 mm Hg). The liquid fatty phase also comprises at least one fatty substance, which is liquid at ambient temperature, also known as, “oils,” with the exception of an ester of an acid and a polyol. The liquid fatty phase may also comprise at least one gelling agent and/or at least one pigment stabilizer that may be present in the composition, provided that they are liquid at ambient temperature and atmospheric pressure. The liquid fatty phase may also comprise a volatile liquid fatty phase and/or a non-volatile fatty phase.

As used herein, the term “non-volatile fatty phase” means any medium that can remain on the skin or lips without evaporating. A non-volatile fatty phase has, for example, a non-zero vapor pressure at ambient temperature and atmospheric pressure, of less than 0.02 mm Hg and, further, for example, less than 10⁻³ mm Hg.

As used herein, the term “volatile fatty phase” means any non-aqueous medium that can evaporate from the skin or lips at ambient temperature and atmospheric pressure. The volatile fatty phase comprises, for example, oils having a vapor pressure at ambient temperature (25° C.) and atmospheric pressure (760 mm Hg) ranging, for example, from 10⁻³ to 300 mm Hg (0.13 Pa to 40 000 Pa), further, for example, ranging from 0.2 to 300 mm Hg (2.66 Pa to 40 000 Pa).

As used herein, the term “hydrocarbon oil” means oils comprising primarily carbon atoms and hydrogen atoms and, for example, at least one chain chosen from alkyl and alkenyl chains, such as alkanes and alkenes, the at least one chain may also comprise at least one group chosen from ethers, esters and carboxylic acid groups.

As used herein, the term “substituted” means further comprising at least one substituent chosen from oxygen, nitrogen and halogen atoms and hydroxyl, ether, oxyalkylene, polyoxyalkylene, carboxyl, amine and amide groups.

As used herein, the term “saccharide” means a monosaccharide or a polysaccharide.

As used herein, the term “polar groups” is well known to a person skilled in the art; the polar groups may, for example, be ionic or non-ionic polar groups chosen from —COOH; —OH; ethylene oxide; propylene oxide; —PO₄; —NHR; and —NR₁R₂, wherein R₁ and R₂, which may be identical or different, may each be chosen from linear and branched C₁ to C₂₀ alkyl and alkoxy radicals or, R₁ and R₂, may optionally form at least one ring.

As used herein, the term “gelling agent” means any compound which increases the viscosity of the medium into which it is incorporated, or which stiffens the medium. As used herein, the term “gelling agent” does not include waxes.

As used herein, the term “wax” means a lipophilic fatty compound which is solid at ambient temperature (25° C.) and exhibits a reversible solid/liquid state change, the compound having a melting temperature of greater than 40° C. and possibly up to 200° C. and having an anisotropic crystalline organization in the solid state. The size of the crystals is such that they diffract and/or, scatter light, thus providing the composition with a turbid, more or less opaque appearance. By bringing the wax to its melting temperature it can be made miscible with the oils and made to form a microscopically homogeneous mixture; however, by bringing the temperature of the mixture back to ambient temperature, the wax is recrystallized in the oils of the mixture. It is this recrystallization in the mixture which is responsible for the decrease in the gloss of the mixture.

As used herein, the term “ungrafted polymer” means a polymer obtained by polymerizing at least one monomer without subsequent reaction of the side chains with another chemical compound.

As used herein, the term “liquid silicone compound” means a silicone compound which is liquid at ambient temperature (25° C.) and atmospheric pressure (760 mm Hg).

Disclosed herein is a cosmetic makeup product comprising at least two compositions wherein,

a first composition comprises a physiologically acceptable medium and

a second composition comprises a mixture of at least one high molecular weight polymer having a weight-average molecular mass of greater than or equal to 200,000 g/mol and at least one low molecular weight non-volatile liquid silicone compound with a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s.

Further disclosed herein is a cosmetic makeup product comprising at least two compositions wherein,

a first composition comprises a physiologically acceptable medium and

a second composition comprises a mixture of at least one high molecular weight polymer having a weight-average molecular mass of greater than or equal to 200,000 g/mol and at least one low molecular weight non-volatile liquid silicone compound with a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s, and

wherein the second composition is applied on top of the first composition.

A makeup product is any product comprising at least one colorant that allows a color to be deposited on a human keratin material, such as the skin, lips and/or epidermal integuments, by applying to the keratin material products, such as lipsticks, rouges, eyeliners, foundations, self-tanning products and/or semi-permanent makeup products (tattoos).

A lip makeup product may, for example, comprise the first composition in the form of a stick or in liquid form and the second composition in fluid form and packaged in a tube. The cosmetic makeup product disclosed herein comprises at least two physiologically acceptable compositions which are packaged separately or together in a single container or in at least two separate or discrete containers.

The compositions may, for example, be packaged separately.

The cosmetic makeup product disclosed herein may, for example, be provided in a form chosen from foundations, blushes, eyeshadows, lipsticks, and products having care properties, for example, eyeliner, concealer and body makeup products (of the tattoo type). For example, the cosmetic makeup product may be a lipstick.

Further disclosed herein is a makeup kit comprising the cosmetic makeup product as defined above, wherein the different compositions are packaged separately and are accompanied by appropriate means of application or are intended for finger application. These means may, for example, be chosen from at least one of fine and coarse brushes, pens, pencils, felts, quills, sponges, tubes, and foam ends. For example, the first composition may be fluid and may be applied using a foam end, while the second composition may be fluid and may be applied using a tube. Further, for example, the first composition may be a stick, while the second composition may be fluid and may be applied using a tube.

The first composition of the cosmetic makeup product disclosed herein may be applied to the keratin material to form a basecoat, over which the second composition is deposited to give a topcoat or finish coat. It is possible to apply under the basecoat an undercoat, whose composition is the same as or different from that of the second coat.

It is also possible to deposit over the first topcoat a second topcoat, whose composition is the same as or different from that of the first topcoat. The resulting makeup may, for example, be a makeup comprising a basecoat and a topcoat.

The first composition may, for example, be provided in a form chosen from foundations, rouges, lipsticks, lipglosses, eyeliners and body makeup products and the second composition may, for example, be provided in a form chosen from care products- and products intended for preserving and/or enhancing at least one of the cosmetic properties of the first composition.

Further disclosed herein is a method of making up the skin and/or lips and/or epidermal derivatives comprising applying to the skin and/or lips and/or epidermal derivatives a cosmetic makeup product as disclosed herein.

Even further disclosed herein is a method of making up the skin and/or lips and/or epidermal integuments of a human being, comprising,

applying to the skin, lips and/or epidermal derivatives, a first coat of the first composition comprising a physiologically acceptable medium and

applying over all or part of the first coat, a second coat, comprising a second composition, wherein the second composition comprises a mixture of at least one high molecular weight polymer having a weight-average molecular mass of greater than or equal to 200,000 g/mol and at least one low molecular weight non-volatile liquid silicone compound with a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s.

For example, the method may comprise,

applying to the skin, lips and/or epidermal integuments of a human, being a first coat of a first composition comprising a physiologically acceptable medium,

leaving the first coat to dry if necessary, and

applying over all or part of the first coat, a second coat, comprising a second composition, wherein the second composition comprises a mixture of at least one high molecular weight polymer having a weight-average molecular mass of greater than or equal to 200,000 g/mol and at least one low molecular weight non-volatile liquid silicone compound with a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s.

This two-coat makeup may be adapted to all products for making up the skin of both the face and the body, the mucosae, such as the lips, the interior of the lower eyelids, and epidermal integuments, such as the nails, eyelashes, hair, eyebrows and even body hairs. The second coat may form designs and may be applied with a pen, pencil and/or any other instrument, such as a sponge, finger, fine or coarse brushes, and quills. This makeup may also be applied to makeup accessories, such as false nails, false eyelashes, hairpieces or else discs or patches which adhere to the skin or lips (of the beauty-spot type).

The cosmetic make-up product additionally provides a made-up substrate comprising a first coat of a first composition comprising a physiologically acceptable medium and a second coat of a second composition deposited over all or part of the first coat and comprising a mixture of at least one high molecular weight polymer having a weight-average molecular mass of greater than or equal to 200,000 g/mol and at least one low molecular weight non-volatile liquid silicone compound with a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s.

Thus, as discussed above, disclosed herein is a cosmetic makeup product comprising at least two compositions wherein a first composition comprises a physiologically acceptable medium and a second composition comprises a mixture of at least one high molecular weight polymer having a weight-average molecular mass of greater than or equal to 200,000 g/mol and at least one low molecular weight non-volatile liquid silicone compound with a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s.

As mentioned above, the dynamic viscosity of the second composition, comprising a mixture of at least one high molecular weight polymer and at least one low molecular weight non-volatile liquid silicone compound as described, is measured with a Mettler RM 180 viscometer. The Mettler RM 180 instrument (Rheomat) can be fitted with different spindles depending on the order of magnitude of the viscosity it is desired to measure. For a viscosity ranging from 0.18 Pa·s to 4.02 Pa·s the instrument is fitted with a 3 spindle. For a viscosity ranging from 1 Pa·s to 24 Pa·s the instrument is fitted with a 4 spindle, and for a viscosity ranging from 8 Pa·s to 122 Pa·s the instrument is fitted with a 5 spindle. The viscosity is read off on the instrument in deflection units (DU). Reference is then made to tables supplied with the measuring instrument, in order to obtain the corresponding value in poises.

The speed at which the spindle rotates is 200 rpm.

From the moment when the spindle is set in rotation, at an imposed constant rotational speed (in this case 200 rpm), the viscosity value of the mixture may vary over time. Measurements are taken at regular intervals until they become constant. The value of the viscosity which has become constant over time is the value retained as being the dynamic viscosity value of the mixture. In accordance with the measuring system 0.75 imposed on the instrument, the measurement of the viscosity is taken after 10 minutes.

The viscosity of the mixture of at least one high molecular weight polymer and at least one low molecular weight non-volatile liquid silicone compound as described may range, for example, from 0.5 Pa·s to 50 Pa·s, further, for example, from 3.5 Pa·s to 25 Pa·s and, even further, for example, from 5 Pa·s to 10 Pa·s.

In one embodiment, the second composition may be transparent.

As used herein, a “transparent composition” is a composition which is transparent to translucent; in other words, a transparent composition transmits at least 40% of light, for example, at least 50% of light, at a wavelength of 750 nm.

The transmission is measured with a Cary 300 Scan UV-Vis spectrophotometer from the company Varian in accordance with the following protocol:

The second composition (above its melting temperature) is poured into a square-section spectrophotometer cuvette with an edge length of 10 mm.

The sample of the second composition is subsequently cooled to 35° C. for 24 hours and then kept in a thermostated chamber at 20° C. for 24 hours.

The light transmitted through the sample of the second composition is then measured in the spectrophotometer by scanning through wavelengths from 700 nm to 800 nm, the measurement being conducted in transmission mode.

A determination is then made of the percentage light transmitted through the sample of the second composition at the wavelength of 750 nm.

The at least one high molecular weight polymer and the at least one low molecular weight non-volatile liquid silicone compound may both be present in an amount of more than 70% by weight, further, for example, more than 80% by weight, further for example, more than 90% by weight and, even further, for example, more than 95% by weight, relative to the total weight of the second composition.

The second composition disclosed herein may, in addition to the at least one high molecular weight polymer and the at least one low molecular weight non-volatile liquid silicone compound, comprise other compounds, which may, for example, be apolar or relatively: non-polar. These apolar or relatively non-polar compounds may, for example, be chosen from silicone compounds, colorants and gelling agents.

For example, the second composition may comprise only apolar and/or relatively non-polar ingredients.

High Molecular Weight Polymer of the Second Composition

When the second composition is liquid it may, for example, comprise from 20% to 50% by weight of the at least one high molecular weight polymer, relative to the total weight of the second composition.

The at least one high molecular weight polymer may, for example, be a silicone polymer.

When the second composition disclosed herein is solid it may, for example, comprise from 2% to 40% by weight of the at least one high molecular weight polymer, relative to the total weight of the second composition.

The at least one high molecular weight polymer may be liquid or solid at ambient temperature and its weight-average molecular mass may, for example, be greater than or equal to 200,000 g/mol, further, for example, may range from 200,000 g/mol to 4,000,000 g/mol, for example, from 200,000 to 2,500,000 g/mol and, even further, for example, from 200,000 g/mol to 2,000,000 g/mol.

The viscosity of the at least one high molecular weight polymer may, for example, range from 1,000 cSt to 10,000,000 cSt, for example, from 10,000 cSt to 5,000,000 CSt, further, for example, from 10,000 cSt to 1,000,000 cSt, for example, from 100,000 cSt to 1,000,000 cSt, and, even further, for example, from 300,000 cSt to 700 000 cSt, measured in accordance with standard ASTM D-445.

The at least one high molecular weight, polymer may, for example, be an ungrafted polymer, i.e., a polymer obtained by polymerizing at least one monomer without subsequent reaction of the side chains with another chemical compound. The at least one high molecular weight polymer may, for example, be chosen from dimethiconols, fluorosilicones, and dimethicones. The at least one high molecular weight may, for example, be a homopolymer.

The at least one high molecular weight polymer may be chosen from silicone polymers corresponding to formula (A) below: 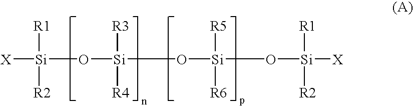
wherein:

R₁, R₂, R₅ and Rr, which may be identical or different, are each chosen from alkyl radicals comprising from 1 to 6 carbon atoms, wherein the carbon atoms are optionally substituted with at least one fluorine atom;

R₃ and R₄, which may be identical or different, are each chosen from alkyl radicals comprising from 1 to 6 carbon atoms and an aryl radical;

X, which may be identical or different, is chosen from alkyl radicals comprising from 1 to 6 carbon atoms, a hydroxyl radical, a vinyl radical, an allyl radical, and alkoxy radicals comprising from 1 to 6 carbon atoms; and

n and p, which may be identical or different, are each chosen such that the at least one high molecular weight polymer has a weight-average molecular mass of greater than or equal to 200,000 g/mol.

In one embodiment, p may be equal to 0.

The polymers of formula (A) wherein R₁ to R₆ are methyl groups and X is a hydroxyl group are dimethiconols. Examples of these dimethiconols include polymers of formula (A) wherein p=0 and n is a number ranging from 2,000 to 40,000, for example, ranging from 3,000 to 30,000. Further examples include polymers having a weight-average molecular mass ranging from 1,500,000 g/mol to 2,000,000 g/mol.

In one embodiment, the at least one high molecular weight polymer is the dimethiconol sold by Dow Corning in a polydimethylsiloxane (5 cSt) under reference D2-9085, the viscosity of the mixture being 1 550 cSt, or the dimethiconol sold by Dow. Coming in a polydimethylsiloxane under reference DC 1503. Further, for example, the at least one high molecular weight polymer may also be a dimethiconol (with a molecular weight of 1 770 000 g/mol) sold by Dow Corning under reference Q2-1403 or Q2-1401, the viscosity of the mixture being 4 000 cSt.

The at least one high molecular weight polymer which can be used in the cosmetic makeup product disclosed herein may be chosen from those polymers wherein:

R₁ to R₆ and X are each a methyl group, such as the product sold under the name SE30 by General Electric and the product sold under the name AK 500000 by Wacker;

R₁ to R₆ and X are each a methyl group and p and n are such that the molecular weight of the at least one high molecular weight polymer is 250,000 g/mol, such as the product sold under the name SILBIONE 70047 V by Rhodia;

R₁ to R₆ are each a methyl group and X is a hydroxyl group, such as the product sold under the name Q2-1401 or Q2-1403 by Dow Corning; and

R₁, R₂, R₅, R₆ and X are each a methyl group, R₃ and R₄ are each an aryl group and n and p, which may be identical or different, are each chosen such that the molecular weight of the polymer is 600,000 g/mol, such as the product sold under the name 761 by Rhône-Poulenc.

The high molecular weight dimethicones disclosed herein include the dimethicones described in U.S. Pat. No. 4,152,416. These polymers are sold for example under references SE30, SE33, SE 54 and SE 76.

The dimethicones disclosed herein may, for example, be chosen from compounds of formula (A) wherein R₁ to R₆ and X are methyls and p=0. The molecular weight of such polymers may range, for example, from 200,000 g/mol to 600,000 g/mol, for example, from 200,000 g/mol to 300,000 g/mol and further, for example, from 240,000 g/mol to 260,000 g/mol.

The dimethicones disclosed herein may be chosen from at least one of polydimethylsiloxanes, (polydimethylsiloxane)(methylvinylsiloxane) copolymers, and poly(dimethylsiloxane)(diphenyl)(methylvinylsiloxane) copolymers.

The high molecular weight fluorosilicones disclosed herein may have a molecular weight ranging, for example, from 200,000 g/mol to 300,000 g/mol and, further, for example, ranging from 240,000 g/mol to 260,000 g/mol.

The at least one high molecular weight polymer may, for example, be introduced into the second composition in the form of a mixture with at least one liquid silicone, the viscosity of the liquid silicone may range from 0.5 cSt to 10,000 cSt, for example, from 0.5 cSt to 500 cSt and, even further, for example, from 1 cSt to 10 cSt.

The at least one liquid silicone may be chosen, for example, from polyalkylsiloxanes, polyarylsiloxanes, and polyalkylarylsiloxanes. The at least one liquid silicone may be a volatile silicone such as a cyclic pblydimethylsiloxane comprising from 3 to 7-(CH₃)₂SiO— units.

The at least one liquid silicone may also be a non-volatile polydimethylsiloxane silicone, for example, with a viscosity ranging from 0.5 cSt to 10,000 cSt, and further, for example, may have a viscosity equal to 5 cSt, for example, the silicone sold under reference DC 200 by Dow Corning.

The viscosity of the at least one liquid silicone is measured in accordance with standard ASTM D-445. The proportion of the at least one high molecular weight polymer in the at least one high molecular weight polymer/at least one liquid silicone mixture may, for example, range from 10/90 to 20/80. The viscosity of the at least one high molecular weight polymer/at least one liquid silicone mixture may, for example, range from 1,000 cSt to 10,000 cSt.

Low Molecular Weight Liquid Silicone Compound of the Second Composition

As described above, the second composition also comprises at least one low molecular weight non-volatile liquid silicone compound with a weight-average molecular mass of less than 200,000 g/mol.

The viscosity of the at least one low molecular weight non-volatile liquid silicone compound at 25° C. may, for example, range from 0.5 cSt to 10,000 cSt, further, for example, from 0.5 cSt to 500 cSt and, even further, for example, from 1 cSt to 10 cSt, measured in accordance with standard ASTM D-445.

The weight-average molecular mass of the at least one low molecular weight non-volatile liquid silicone compound may, for example, range from 400 g/mol to 200,000 g/mol, further, for example, from 4,000 g/mol to 100,000 g/mol, further, for example, from 4,000 g/mol to 20,000 g/mol, further, for example, from 400 g/mol to 2,000 g/mol and, even further, for example, from 400 g/mol to 1,000 g/mol.

The at least one low molecular weight non-volatile liquid silicone compound may be chosen from polyalkylsiloxanes, polyarylsiloxanes, and polyalkylarylsiloxanes.

The at least one low molecular weight non-volatile liquid silicone compound may also be chosen from:
non-volatile linear and branched polydimethylsiloxanes (PDMS);
polydimethylsiloxanes comprising at least one group chosen from alkyl, alkoxy and phenyl groups, pendantly or at the silicone chain end, wherein the at least one groups comprises from 2 to 24 carbon atoms; and
phenylsilicones, such as phenyltrimethicones, phenyldimethicones, phenyltrimethylsiloxydiphenylsiloxanes, diphenyldimethicones, diphenylmethyl-diphenyltrisiloxanes and 2-phenylethyl trimethylsiloxysilicates.

The at least one low molecular weight non-volatile liquid silicone compound may be chosen from silicones modified with at least one group chosen from aliphatic and aromatic groups, optionally fluorinated, and functional groups such as hydroxyl, thiol and amine groups.

The polyalkylsiloxanes may, for example, be chosen from at least one of polydimethylsiloxanes, (polydimethylsiloxane)(methylvinylsiloxane) copolymers, poly(dimethylsiloxane)(diphenyl)siloxanes, and poly(dimethylsiloxane)-(diphenyl)(methylvinylsiloxane) copolymers.

For example, a polydimethylsiloxane such as that sold under the name DC 200 with a viscosity of 5 cSt and a molecular weight of 800, sold by Dow Corning can be used in the second composition disclosed herein.

The second composition may, for example, comprise a mixture of a polydimethylsiloxane with a molecular mass ranging from 200,000 g/mol to 300,000 g/mol and a polydimethylsiloxane with a molecular mass ranging from 400 g/mol to 1,000 g/mol.

The proportion by mass of the high molecular weight silicone compound/liquid silicone compound may, for example, range from 20/80 to 60/40, further, for example, from 35/65 to 45/55.

Volatile Oil in the Second Composition

The second composition may also optionally comprise at least one volatile oil.

The at least one volatile oil may be chosen from hydrocarbon oils and silicone oils optionally comprising at least one group chosen from alkyl and alkoxy groups, pendantly or at the end of the silicone chain.

The at least one volatile oil may, for example, be cosmetic oils chosen from oils having a flashpoint ranging from 40° C. to 100° C. Moreover, the at least one volatile oil may have a boiling temperature at atmospheric pressure of less than 220° C. and, further, for example, less than 210° C., ranging, for example, from 110 to 210° C. For example, the at least one volatile oil is not a monoalcohol comprising 7 or more carbon atoms.

For example, the at least one volatile oil may be chosen from linear and cyclic silicone oils having a viscosity at ambient temperature of less than 8 cSt and comprising, for example, from 2 to 7 silicon atoms, these silicones optionally comprising at least one group chosen from alkyl and alkoxy groups comprising from 1 to 10 carbon atoms. The volatile silicone oils may, for example, be chosen from at least one of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, and dodecamethylpentasiloxane.

The at least one volatile oil may also be chosen from volatile hydrocarbon oils comprising from 8 to 16 carbon atoms, for example, branched C₈-C₁₆ alkanes, such as C₈-C₁₆ isoalkanes (also called isoparaffins), isododecane, isodecane, isohexadecane, and, for example, the oils sold under the trade names ISOPAR and PERMETHYL, and branched C₈-C₁₆ esters such as isohexyl neopentanoate.

For example, the at least one volatile oil may be chosen from isododecane (Permethyl 99 A) and C₈-C₁₆ isoparaffins such as Isopar L, E, G or H, optionally in combination with decamethyltetrasiloxane or with cyclopentasiloxane.

Generally speaking, the amount of the at least one volatile oil used is an amount sufficient to improve the spreading qualities of the second composition. This amount will be adapted by the person skilled in the art in accordance with the intensity of the desired properties.

The amount of the at least one volatile oil is chosen so as not to lessen the gloss of the second composition. In one embodiment, the second composition does not comprise the at least one volatile oil.

Non-Volatile Liquid Phase of the Second Composition

The second composition may also, for example, comprise from 15 to 90% by weight of at least one non-volatile liquid phase.

As defined above, the at least one non-volatile liquid phase is any medium that can remain on the skin or the lips for several hours. The at least one non-volatile liquid phase has, for example, a non-zero vapor pressure at ambient temperature and atmospheric pressure, for example, of less than 0.02 mm. Hg (2.66 Pa) and, further, for example, of less than 10⁻³ mm Hg (0.13 Pa).

The at least one non-volatile liquid phase of the second composition may, for example, be apolar or relatively non-polar and, for example, comprise at least one non-volatile oil chosen from hydrocarbon oils, silicone oils and liquid fluoro oils.

The at least one non-volatile oil may be chosen, for example, from polydimethylsiloxanes; alkyldimethicones; polyphenylmethylsiloxanes, such as phenyldimethicones and phenyltrimethicones; and silicones modified with at least one group chosen from aliphatic and aromatic groups, optionally fluorinated, and functional groups such as hydroxyl, thiol and amine groups.

The at least one non-volatile oil may be chosen from silicones of formula (I): 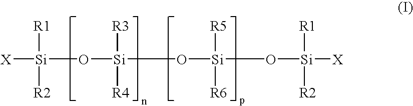
wherein:

R₁, R₂, R₅ and R₆, which may be identical or different, are each chosen from alkyl radicals comprising from 1 to 6 carbon atoms;

R₃ and R₄, which may be identical or different, are each chosen from alkyl radicals comprising from 1 to 6 carbon atoms and aryl radicals;

X, which may be identical or different, is chosen from alkyl radicals comprising from 1 to 6 carbon atoms, a hydroxyl radical and a vinyl radical; and

n and p, which may be identical or different, are chosen so as to give the silicone compound a weight-average molecular mass of less than 200 000 g/mol, for example, less than 150,000 g/mol and, further, for example, less than 100,000 g/mol.

The non-volatile silicone of formula (I) chosen may be, for example, a polydimethylsiloxane with a viscosity ranging from 0.5 cSt to 60,000 cSt, further, for example, ranging from 0.5 cSt to 0.10 000 cSt, for example, the product DC 200 with a viscosity of 350 cSt, sold by Dow Corning.

The at least one non-volatile liquid phase of the second composition may, for example, comprise at least one non-volatile oil chosen from fluoro oils of formula (II): 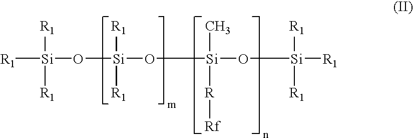
wherein:

R is chosen from linear and branched alkylenyl groups comprising from 1 to 6 carbon atoms, for example, a divalent methylenyl, ethylenyl, propylenyl or butylenyl group;

Rf is chosen from fluoroalkyl radicals, for example, a perfluoroalkyl radical, comprising from 1 to 9 carbon atoms, for example, 1 to 4 carbon atoms;

R₁, which may be identical or different, is chosen from C₁-C₂₀, for example, C₁-C₄, alkyl radicals, a hydroxyl radical and a phenyl radical;

m is a number ranging from 0 to 150, for example, from 20 to 100; and

n is a number ranging from 1 to 300, for example, from 1 to 100.

For example, oils of formula (II) wherein R₁ is a methyl, R is an ethyl and Rf is CF₃ can be used. Fluorosilicone compounds of formula (II) which may be used are those sold by Shin Etsu under the names X22-819, X22-820, X22-821 and X22-822 and FL-100.

Examples of fluoro oils include fluorinated polyethers chosen from compounds of formula (III):
R₆—(CF₂—CFR₃—CF₂O)ₚ—(CFR₄—CF₂—O)_{q}—(CFR₅—O)ᵣR₇ (III)
wherein:

R₃ to R₆, which may be identical or different, are each a monovalent radical chosen from —F, —(CF₂)ₙ—CF₃ and —O—(CF₂)ₙ—CF₃;

R₇ is a monovalent radical chosen from —F and —(CF₂)ₙ—CF₃, wherein n is a number ranging from 0 to 4 inclusive; and

p ranges from 0 to 600, q from 0 to 860 and r from 0 to 1 500, p, q and r are integers chosen such that the weight-average molecular mass of the compound ranges from 500 g/mol to 100,000 g/mol, for example, from 500 g/mol to 10,000 g/mol.

The fluoro oils may also be chosen from fluorinated alkanes chosen in turn from at least one of C₂-C₅₀, for example, from C₅-C₃₀, fluoroalkanes and perfluoroalkanes, such as perfluorodecalin, perfluoroadamantane and bromoperfluorooctyl.

Gelling Agent in the Second Composition

In one embodiment, the second composition may comprise at least one gelling agent for the at least one liquid fatty phase, the at least one gelling agent may be chosen from polymeric and mineral gelling agents.

The at least one gelling agent increases the viscosity of the at least one liquid fatty phase or stiffens it. The at least one gelling agent disclosed herein does not include waxes.

The at least one gelling agent may be chosen from ethylene homopolymers and copolymers whose weight-average molecular mass ranges from 300 g/mol to 500,000 g/mol and, for example, from 500 g/mol to 100,000 g/mol.

The at least one gelling agent may be chosen from olefin copolymers of controlled crystallization, as described in Patent Application No. EP-A-1 034 776, such as, for example, the ethylene/octene copolymer sold under the name ENGAGE 8400 by DuPont de Nemours.

This olefin copolymer or copolymers may, for example, be present in an amount ranging from 0.5% to 20% by weight, and, for example, from 1% to 10% by weight, relative to the total weight of the second composition.

In another embodiment, a polycaprolactone may be used as the at least one gelling agent.

The polycaprolactone may, for example, be chosen from ε-caprolactone homopolymers. Homopolymerization may be initiated with a diol, for example, a diol comprising from 2 to 10 atoms, such as diethylene glycol, 1,4-butanediol and neopentyl glycol.

It is possible, for example, to use polycaprolactones with a molecular weight ranging from 300 g/mol to 2,000 g/mol, for example, those sold under the name CAPA 2125 by Solvay.

The polycaprolactones may be present in the second composition in an amount ranging from 0.1% to 30% by weight, for example, from 0.5% to 25% by weight and, further, for example, from 1% to 20% by weight, and even further, for example, from 3% to 15% by weight, relative to the total weight of the second composition.

In another embodiment the at least one gelling agent present in the composition disclosed herein is an amorphous polymer formed by polymerizing an olefin as described in Patent Application No. EP 1 002 528, the disclosure of which relates to the amorphous polymer is specifically incorporated herein by reference. The olefin may, for example, be an elastomeric ethylenically unsaturated monomer.

Examples of olefins include ethylenic hydrocarbon monomers, comprising, for example, one or two ethylenic unsaturations and comprising from 2 to 5 carbon atoms, such as ethylene, propylene, butadiene and isoprene.

As used herein, an “amorphous polymer” is a polymer which does not have a crystalline form.

The at least one gelling agent may be chosen from amorphous polymeric gelling agents. The amorphous polymeric gelling agents may, for example, be chosen from at least one of diblock, triblock, multiblock, radial and star copolymers, for example, triblock, multiblock, radial and star copolymers.

Polymeric gelling agents of this kind are described in Patent Application No. US-A-2002/005562 and in U.S. Pat. No. 5,221,534.

The polymeric gelling agents may, for example, be an amorphous block copolymer of styrene and an olefin.

The polymeric gelling agents may, for example, be hydrogenated in order to reduce the ethylenic unsaturations which remain after the polymerization of the monomers.

For example, the polymeric gelling agents are optionally hydrogenated copolymers comprising styrene blocks and ethylene/C₃-C₄ alkylene blocks.

The polymeric gelling agents may, for example, be a triblock copolymer, for example, hydrogenated, chosen from styrene-ethylene/propylene-styrene copolymers, styrene-ethylene/butadiene-styrene copolymers, styrene-isoprene-styrene copolymers and styrene-butadiene-styrene copolymers. Triblock polymers are sold, for example, under the names KRATON G1650E, KRATON G1652, KRATON D1101, KRATON D1102 and KRATON D1160 by Kraton.

The amorphous polymeric gelling agents may be present in an amount ranging, for example, from 0.05% to 5% by weight, and for example, ranging from 0.1% to 3% by weight, and, further, for example, ranging from 0.2% to 2% by weight, relative to the total weight of the second composition.

The polymer particles dispersed in the organic phase and the polymeric gelling agents may, for example, be present in the second composition in an amount such that the weight ratio of dispersed and surface-stabilized polymer particles to polymeric gelling agent ranges, for example, from 10:1 to 30:1, for example, from 15:1 to 25:1, and further, for example, from 18:1 to 22:1.

In another embodiment, the at least one gelling agent may, for example, be chosen from:

pyrogenic silica, optionally hydrophobically treated on its surface, with a particle size of less than 10 microns and

optionally modified clays, such as hectorites modified with a C₁₀ to C₂₂ fatty acid ammonium chloride, such as hectorite modified with distearyldimethylammonium chloride.

The organic-phase gelling agents may, for example, be chosen from:

partly and totally crosslinked elastomeric organopolysiloxanes of three-dimensional structure, such as those sold under the name KSG6, KSG16 and KSG18 by Shin-Etsu, TREFIL E-505C and TREFIL E-506C by Dow Corning, GRANSIL SR-CYC, SR DMF10, SR-DC556, SR 5CYC gel, SR DMF 10 gel and SR DC 556 gel by Grant Industries, and SF 1204 and JK 113 by General Electric and

galactomannans comprising, for example, from one to six and, further, for example, from two to four hydroxyl groups per monosaccharide unit, which are substituted by at least one chain chosen from saturated and unsaturated alkyl chains, such as guar gum alkylated with at least one chain chosen from C₁ to C₆ and, for example, C₁ to C₃ alkyl chains, and, further, for example, the ethylated guar having a degree of substitution ranging from 2 to 3, such as that sold by Aqualon under the name N-Hance-AG.

Wax in the Second Composition

The second composition may also, for example, comprise at least one wax, for example, when it is in solid form.

The at least one wax may be present in an amount ranging, for example, from 2% to 30% by weight, further, for example, from 5% to 20% by weight and, even further, for example, from 5% to 15% by weight, relative to the total weight of the second composition.

For example, the at least one wax may be chosen from linear hydrocarbon waxes. The melting point of the at least one wax may, for example, be greater than 35° C., further, for example, greater than 55° C., even further, for example, greater than 80° C.

The linear hydrocarbon waxes may be chosen from, for example, substituted linear alkanes, unsubstituted linear alkanes, unsubstituted linear alkenes, and substituted linear alkenes, an unsubstituted compound being composed solely of carbon and hydrogen, and wherein the substituents mentioned above do not contain any carbon atoms.

The linear hydrocarbon-waxes include polymers and copolymers of ethylene with a molecular weight ranging from 400 g/mol to 800 g/mol, for example POLYWAX 500 and POLYWAX 400, sold by New Phase Technologies.

Linear hydrocarbon waxes include linear paraffin waxes, such as paraffins S&P 206, S&P 173 and S&P 434 from Strahl & Pitsch.

Linear hydrocarbon waxes include long-chain linear alcohols, such as the products comprising a mixture of polyethylene and alcohols comprising from 20 to 50 carbon atoms, for example, PERFORMACOL 425 and PERFORMACOL 550 (mixture in 20/80 proportions) sold by New Phase Technologies.

The second composition may, for example, comprise at least one wax chosen from silicone waxes, such as a dimethicone comprising alkyl groups at the chain end. These alkyl groups may, for example, comprise more than 18 carbon atoms, further, for example, ranging from 20 to 50 carbon atoms, and, even further, for example, ranging from 30 to 45 carbon atoms.

The silicone waxes correspond, for example, to the formulae (IV) or (V) below: 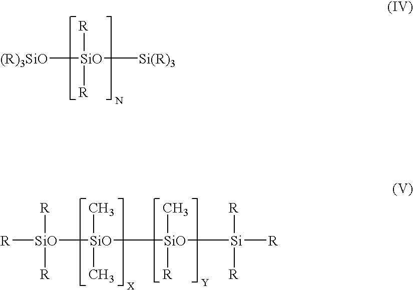
wherein R, which may be identical or different, is an alkyl, X is greater than or equal to zero, and N and Y, which may be identical or different, are greater than or equal to one.

R comprises 1 to 50 carbon atoms, subject to the proviso that the compound is solid at ambient temperature.

Examples of silicone waxes include:
C₂₀-C₂₄ alkyl methicones, C₂₄-C₂₈ alkyl dimethicones, C₂₀-C₂₄ alkyl dimethicones and C₂₄-C₂₈ alkyl dimethicones, sold by Archimica Fine Chemicals as SILCARE 41M40, SILCARE 41M50, SILCARE 41M70 and SILCARE 41M80;
stearyl dimethicones sold under the name SILCARE 41M65 by Archimica, and DC-2503 by Dow Corning;
stearoxytrimethylsilanes sold as SILCARE 41M71 and DC-580;
the products ABIL WAX 9810, 9800 or 2440 from Wacker-Chemie GmbH; and
C₃₀-C₄₅ alkyl methicones sold by Dow Corning as AMS-C30 Wax, and C₃₀-C₄₅ alkyl dimethicones sold as SF1642 and SF-1632 by General Electric.
   Colorant

The second composition of the cosmetic makeup product disclosed herein may comprise at least one colorant, which may be chosen from water-soluble and fat-soluble dyes, pigments, and nacres.

The pigments are chosen from white and colored, organic and mineral particles which are insoluble in the liquid organic phase and are intended for coloring and/or opacifying the first composition.

The nacres are chosen from iridescent particles, produced, for example, by certain molluscs within their shell, or else synthesized, which are insoluble in the medium of the first composition.

The dyes are chosen from compounds, generally organic compounds, which are soluble in fatty substances, such as oils, and in an aqueous-alcoholic phase.

The fat-soluble dyes may, for example, be chosen from Sudan Red, D&C Red No. 17, D&C Green No. 6, β-carotene, soya oil, Sudan Brown, D&C Yellow No. 11, D&C Violet No. 2, D&C Orange No. 5, quinoline yellow, annatto and bromo acids.

The water-soluble dyes may, for example, be chosen from beet juice, methylene blue and caramel.

The pigments may be chosen from white and colored pigments, mineral and organic pigments, and interference pigments and non-interference pigments. Examples of mineral pigments include titanium dioxide, optionally with surface treatment, zirconium oxide and cerium oxide, and zinc oxide, iron oxide (black, yellow brown and red) and chromium oxide, manganese violet, ultramarine blue, chromium hydrate and ferric blue. Examples of organic pigments include carbon black, organic lake pigments of barium, strontium, calcium and aluminium, including those certified by the U.S. Food and Drug Administration (FDA) (examples D&C or FD&C) and those exempt from FDA certification, such as lakes based on cochineal carmine.

The nacres and nacreous pigments may be chosen from white nacreous pigments such as titanium-covered mica and bismuth oxychloride, colored nacreous pigments such as titanium mica with iron oxides, titanium mica with, for example, ferric blue and chromium oxide, titanium mica with an organic pigment of the aforementioned type, and also nacreous pigments based on bismuth oxychloride. Pigments having goniochromatic properties and metal effect pigments may also be used, such as those described in Patent Application No. FR 0 209 246, the disclosure that relates to such pigments is specifically incorporated herein by reference.

The second composition may, but need not necessarily, comprise at least one colorant.

The at least one colorant may be present in an amount ranging, for example, from 0.001% to 60% by weight, further, for example, from 0.01% to 50% by weight, and, even further, for example, from 0.1% to 40% by weight, relative to the total weight of the first or second composition.

The at least one colorant and/or at least one filler may additionally be present in the form of a “particle paste”.

Additives and Pharmaceuticals of the First or Second Composition

The cosmetic make-up product may further comprise at least one active agent chosen from cosmetic and dermatological active agents such as those conventionally employed.

The at least one active agent may, for example, be chosen from moisturizers, vitamins, essential fatty acids, sphingolipids and sunscreens. The at least one active agent is used in an amount which is customary for the person skilled in the art, and, for example, may be present in an amount ranging from 0% to 20% by weight and, further, for example, from 0.001% to 1.5% by weight, relative to the total weight of the first or second composition.

The first or second composition may further comprise any other additive commonly used in such compositions, such as water, antioxidants, perfumes, preservatives and essential oils.

The person skilled in the art will of course take care to select this or these optional complementary compound(s), and/or their amount, such that the advantageous properties of the compositions disclosed herein are not, or not substantially, adversely affected by the intended addition(s).

For example, the compositions disclosed herein may be prepared in customary manner by the person skilled in the art. They may be provided in a form chosen from cast products, and for example in the form of sticks and crayons, and dishes, which can be used by direct contact or with a sponge. For example, the composition may be provided in form chosen from cast foundations, cast blushes and cast eyeshadows, lipsticks, care bases and care balms for the lips and concealer products. The compositions may also be provided in a form chosen from soft pastes, gel, more or less fluid creams, and liquids, which may be packaged in a tube. The compositions may therefore be foundations, lipsticks, sun products and/or skin coloring products.

The compositions disclosed herein may, for example, each be anhydrous and in that case, the compositions can comprise a total content of water of no more than 5% by weight, relative to the total weight of the composition.

When the compositions are used for topical application, they may, for example, be provided in the form of cosmetic, derrnatological, hygiene and/or pharmaceutical compositions for the protection, treatment and/or care of the face, neck, hands and/or body (for example care cream, sun oil, body gel); makeup compositions (for example a makeup gel, cream or stick); artificial tanning compositions; and skin protection compositions.

The compositions disclosed herein may, for example, be in a form chosen from dermatological and care compositions for the skin and/or epidermal derivatives; sun protection compositions; and body hygiene compositions, for example, in the form of a deodorant. If the compositions are in the form of a deodorant, they may, for example, be provided in an uncolored form. The compositions may therefore be used as a care base for the skin, epidermal derivatives and/or lips (lip balms, protecting the lips from cold and/or sun and/or wind, or care cream for the skin, nails and/or hair).

The compositions disclosed herein may, for example, be lipsticks in the form of a stick or in fluid form.

The compositions disclosed herein must be cosmetically or dermatologically acceptable; that is, it must comprise a non-toxic physiologically acceptable medium which can be applied to the skin, epidermal derivatives and/or lips of the face of human beings. A “cosmetically acceptable composition,” as used herein, is a composition whose appearance, odor and feel are pleasant.

Each composition of the two-coat makeup product disclosed herein may be present in any pharmaceutical form which is normally used for topical application, and, for example, in a form chosen from oily and aqueous solutions, oily and aqueous gels, oil-in-water and water-in-oil emulsions, multiple emulsions, dispersions of oil in water which are mediated by vesicles, wherein the vesicles are situated at the oil/water interface, and powders. Each composition may be fluid or solid.

For example, the first or the second composition, or both, may have a continuous fatty phase and may, for example, be in anhydrous form, and may comprise no more than 5% of water, for example, no more than 1% of water, relative to the total weight of the first or second composition. For example, the cosmetic makeup product disclosed herein may be in an anhydrous form.

Each first and second composition may be in a form chosen, for example, from lotions; creams; ointments; soft pastes; salves; cast and molded solids, for example, stick form or dish form; and compacted solids.

Each composition may, for example, be in the form of a more or less rigid stick.

Each composition may be packaged separately in a single container, for example in a twin-compartment pen, the base composition being delivered by one end of the pen and the top composition being delivered by the other end of the pen, each end being closed, for example, tightly, by means of a cap.

The composition which is applied in a first coat may, for example, be in solid form, and may allow more practical application, better temporal stability and temperature stability of the composition, and a precise line of the makeup, which is highly desirable in the case of a lipstick or an eyeliner.

The cosmetic makeup product disclosed herein may, for example, be used for making up the skin and/or lips and/or epidermal derivatives, in accordance with the nature of the ingredients employed. For example, the cosmetic product may be in a form chosen from solid foundations, lipsticks and lipstick pastes, concealer products, eye contour products, eyeliners, mascaras, eye shadows, body makeup products, and skin-coloring products.

The cosmetic makeup product disclosed herein may, for example, be a lipstick.

For example, the first and/or the second composition may be in solid form.

The topcoat may, for example, have at least one of the following properties: care, gloss and transparency.

The cosmetic makeup product disclosed herein may be provided in a form chosen from lip products, foundations, tattoos, blushes, and eyeshadows, wherein the cosmetic makeup product comprises the first and second compositions disclosed herein.

The compositions disclosed herein may be obtained by heating the various constituents to the melting temperature of the highest waxes, then casting the melted mixture in a mold (dish or glove finger). The may also be obtained by extrusion as described in Patent Application No. EP-A-0 667 146.

When the physiologically acceptable medium of the first and/or second composition comprises a liquid organic phase, this medium may, for example, comprise water dispersed or emulsified in the liquid organic phase.

For example, the compositions disclosed herein may be prepared in customary manner by the person skilled in the art. They may be in the form chosen from cast products and, for example, sticks and wands, soft pastes in a pot, and dishes which can be used by direct contact and/or with a sponge. For example, the compositions, which may be identical or different, may be in a form chosen from cast foundations; blushes; cast eyeshadows, colored, for example; lipsticks; lip glosses; and concealers. They may also each be present in a form chosen from soft pastes; gels; and more or less fluid creams. In that case, the compositions may be in a form chosen from fluids and pastelike foundations and lipsticks, lip glosses, sun products and skin coloring products, eyeliners and body makeup products. The compositions may also exhibit at least one care property, in which case they may be in the form of a lipcare base or balm.

First Composition

In one embodiment, the first composition disclosed herein comprises polymer particles dispersed in a liquid fatty phase.

Dispersed Polymer Particles

The polymer particles are solid and may be insoluble in the liquid organic phase of the first composition even at its softening temperature, in contradistinction to a wax, even a wax of polymeric origin, which is soluble in the liquid organic phase (or fatty phase) at its melting temperature. In addition, it allows the formation of a deposit, for example, a film-forming deposit, which is continuous and homogeneous and/or it is characterized by the interpenetration of the polymeric chains. With a wax, even one obtained by polymerization, melting in the liquid organic phase is followed by recrystallization. It is this recrystallization which is responsible, for example, for the loss of gloss of the composition.

To optimize the non-transfer properties, the amount of the polymer particles are selected as a function of the amount of colorants and/or actives and/or oils present in the first composition. For example, the amount of polymer particles may be greater than 5% by weight (in terms of active substance), relative to the total weight of the first composition.

One advantage of using a dispersion of polymer particles in the first composition is that these particles remain in the state of elementary particles, without forming agglomerates, in the fatty phase. Another advantage of the polymer dispersion is the possibility of obtaining highly fluid compositions (of the order of 130 centipoises), even in the presence of a high proportion of polymer.

Yet another advantage of a polymer dispersion of this kind is that the polymer particle size can be calibrated at will and its size “polydispersity” modified during the synthesis. It is thereby possible to obtain very small-sized particles which are invisible to the naked eye when in the composition and when applied to the skin, lips and/or epidermal derivatives.

A further advantage of the polymer dispersion of the first composition is the possibility of varying the glass transition temperature (T_{g}) of the polymer or polymeric system (polymer and plasticizer-type additive) and hence of passing from a hard polymer to a more or less soft polymer, thereby allowing the mechanical properties of the composition to be regulated as a function of the intended application and, for example, of the film deposited.

The first composition, therefore, for example, comprises a dispersion of polymer particles, generally spherical particles of at least one polymer, in a physiologically acceptable liquid organic phase. These dispersions may, for example, be in the form of polymer nanoparticles in stable dispersion in the liquid organic phase. The nanoparticles may, for example, have an average size ranging from 5 nm to 800 nm and, further, for example, from 50 to 500 nm. It is possible, however, to obtain polymer particle sizes ranging up to 1 μm.

The polymer particles in dispersion may, for example, be insoluble in water-soluble alcohols such as ethanol, for example.

The polymers in dispersion that can be used in the first composition may have, for example, a molecular weight ranging from 2,000 g/mol to 10,000,000 g/mol, and a T_{g} ranging from −100° C. to 300° C. and, for example, from −50° C. to 100° C., and, further, for example, from −10° C. to 50° C.

When the polymers in dispersion have a glass transition temperature which is too high for the desired application it is possible to combine it with a plasticizer so as to lower this temperature of the mixture used. The plasticizer may be chosen from the plasticizers commonly used in the field of application, and, for example, from compounds able to act as solvents for the polymer. It is additionally possible to use coalescence agents in order to promote the formation of a continuous and homogeneous deposit of the polymer.

The coalescence agents or plasticizers which can be used are those referred to in FR-A-2 782 917.

It is possible to use filmable polymers, for example, having a low T_{g}, less than or equal to the temperature of the skin, and, for example, less than or equal to 40° C.

The polymer used may, for example, be filmable, that is, it is able, alone or in combination with a plasticizer, to form an isolatable film. It is possible, however, to use a non-filmable polymer.

As used herein, a “non-filmable polymer” is a polymer which is not capable on its own of forming an isolatable film. In combination with a non-volatile compound of the oil type, this polymer allows a continuous and homogeneous deposit to be formed on the skin and/or lips.

Examples of filmable polymers include radical, acrylic and vinyl homopolymers and copolymers, for example, having a T_{g} of less than or equal to 40° C. and, for example, ranging from −10° C. to 30° C., which may used alone or in a mixture.

Examples of non-filmable polymers include radical, vinyl and acrylic homopolymers or copolymers, optionally crosslinked, having a T_{g} of, for example, greater than 40° C. and, for example, ranging from 45° C. to 150° C., which may be used alone or in a mixture.

A radical polymer is a polymer obtained by polymerizing monomers comprising unsaturation, for example, ethylenic unsaturation, each monomer being capable, of undergoing homopolymerization (in contrast to polycondensates). The radical polymers may, for example, be vinyl polymers or copolymers, for example, acrylic polymers.

The acrylic polymers may result from the polymerization of ethylenically unsaturated monomers comprising at least one acid group and/or esters of these acid monomers and/or amides of these acid monomers.

As a monomer bearing at least one acid group it is possible to use α,β-ethylenic unsaturated carboxylic acids such as acrylic acid, methacrylic acid, crotonic acid, maleic acid and itaconic acid. For example, (meth)acrylic acid and crotonic acid may be used, and, further, for example, (meth)acrylic acid may be also be used.

The esters of acid monomers may, for example, be chosen from esters of (meth)acrylic acid (also called (meth)acrylates), such as alkyl (meth)acrylates, for example, C₁-C₂₀, such as C₁-C₈, alkyl (meth)acrylates, aryl (meth)acrylates, for example, C₆-C₁₀ aryl (meth)acrylates, and hydroxyalkyl (meth)acrylates, for example, C₂-C₆ hydroxyalkyl (meth)acrylates. Examples of alkyl (meth)acrylates include methyl, ethyl, butyl, isobutyl, 2-ethylhexyl and lauryl (meth)acrylate. Examples of hydroxyalkyl (meth)acrylates include hydroxyethyl (meth)acrylate and 2-hydroxypropyl (meth)acrylate. Examples of aryl (meth)acrylates include benzyl acrylate and phenyl acrylate.

Further, for example, the esters of (meth)acrylic acid may be chosen from alkyl (meth)acrylates.

The radical polymer may, for example, be chosen from copolymers of (meth)acrylic acid and alkyl (meth)acrylate, for example, C₁-C₄ alkyl (meth)acrylate. It is possible to use, for example, methyl acrylates optionally copolymerized with acrylic acid.

Examples of the amides of the acid monomers include (meth)acrylamides, and, for example, N-alkyl (meth)acrylamides, for example, N-C₂-C₁₂ alkyl(meth)acrylamides, such as N-ethylacrylamide, —N-t-butylacrylamide and N-octylacrylamide, and N-di(C₁-C₄)alkyl(meth)acrylamides.

The acrylic polymers may likewise result from the polymerization of ethylenically unsaturated monomers comprising at least one amine group, in free form or in partly or totally neutralized form, or in partly or totally quaternized form. Such monomers may be, for example, dimethylaminoethyl (meth)acrylate, dimethylaminoethylmethacrylamide, vinylamine, vinylpyridine and diallydimethylammonium chloride.

The vinyl polymers may likewise result from the homopolymerization or copolymerization of at least one monomer chosen from vinyl esters and styrenic monomers. For example, these monomers may be polymerized with acid monomers and/or their esters and/or their amides, such as those mentioned above. Examples of vinyl esters include vinyl acetate, vinyl propionate, vinyl neodecanoate, vinyl pivalate, vinyl benzoate and vinyl t-butylbenzoate. Examples of styrenic monomers include styrene and alpha-methylstyrene.

The list of monomers given is not limitative and it is possible to use any monomer known to the person skilled in the art which falls within the categories of acrylic monomers and vinyl monomers (including monomers modified with a silicone chain).

Examples of other vinyl monomers which can be used include the following:

N-vinylpyrrolidone, vinylcaprolactam, vinyl-N-(C₁-C₆)alkylpyrroles, vinyloxazoles, vinylthiazoles, vinylpyrimidines and vinylimidazoles; and

olefins such as ethylene, propylene, butylene, isoprene and butadiene.

The vinyl polymer may be crosslinked using at least one difunctional monomer, including, for example, at least two ethylenic unsaturations, such as ethylene glycol dimethacrylate and diallyl phthalate.

Without limitation, the polymer particles in the dispersion of the first composition may be chosen from at least one of the following polymers and copolymers: polyurethanes, acrylic polyurethanes, polyureas, polyurea-polyurethanes, polyester-polyurethanes, polyether-polyurethanes, polyesters, polyester amides, fatty-chain polyesters, alkyds; acrylic and vinyl polymers and copolymers; acrylic-silicone copolymers; polyacrylamides; silicone polymers such as silicone polyurethanes and silicone acrylics, and fluoro polymers.

The polymer particles in dispersion in the organic liquid phase may be present in an amount ranging from 2 to 40% by weight, for example, from 5 to 40% by weight, further, for example, from 5 to 35% by weight, and, even further, for example, from 8 to 30% by weight, relative to the dry-matter content of the first composition.

In one embodiment, the polymer particles in dispersion are surface-stabilized by at least one stabilizer which is solid at ambient temperature. In that case, the amount of the dispersion in terms of dry matter represents the total amount of polymer+stabilizer, subject to the proviso that the amount of polymer in the dispersion may not be less than 5%.

For example, a dispersion of filmable polymers may be used, wherein the particles are dispersed in at least one volatile oil.

Polymer Particle Stabilizer

The polymer particles may, for example, be surface-stabilized by means of at least one stabilizer which may be chosen from block polymer, graft polymers and random polymers. Stabilization can be effected by any known means, for example, by direct addition of the block polymer, graft polymer and/or random polymer during the polymerization.

The at least one stabilizer may also, for example, be present in the mixture before polymerization of the polymer. However, it is also possible to add it continuously, for example, when the monomers are also added continuously.

For example, from 2% to 30% by weight of the at least one stabilizer relative to the initial mixture of monomers can be used, and, for example, 5% to 20% by weight.

When graft polymer and/or block polymers are used as the at least one stabilizer, the synthesis solvent is chosen such that at least some of the grafts or blocks in the at least one stabilizing polymer is soluble in the solvent, the remainder of the grafts or blocks are not soluble in the solvent. The at least one stabilizing polymer used during the polymerization should be soluble or dispersible in the synthesis solvent. Furthermore, the at least one stabilizer may, for example, be chosen from polymers whose insoluble blocks or grafts have a certain affinity for the polymer formed during the polymerization.

Examples of the graft polymers include silicone polymers grafted with a hydrocarbon chain and hydrocarbon polymers grafted with a silicone chain.

Thus, block or graft block copolymers comprising at least one block of polyorganosiloxane and at least one block of a radical polymer can be used, such as graft copolymers of acrylic/silicone, which can be used, for example, when the non-aqueous medium is silicone-based.

It is also possible to use block or graft block copolymers comprising at least one block of polyorganosiloxane and at least one polyether. The polyorganopolysiloxane-block can be, for example, a polydimethylsiloxane or alternatively a poly(C₂-C₁₈)-alkylmethylsiloxane; the polyether block can be a poly(C₂-C₁₈)alkylene, for example, polyoxyethylene and/or polyoxypropylene. For example, it is possible to use dimethicone copolyols or (C₂-C₁₈)alkyldimethicone copolyols such as those sold under the name Dow Corning 3225C by the company Dow Corning, and lauryl methicones such as those sold under the name Dow Corning Q2-5200 by the company Dow Corning.

Examples of block or graft block copolymers which may used are those comprising at least one block resulting from the polymerization of at least one ethylenic monomer comprising at least one optionally conjugated ethylenic bond, such as ethylene or dienes, for example, butadiene and isoprene, and at least one block of a vinyl polymer, for example, a styrenic polymer. When the ethylenic monomer comprises at least one optionally conjugated ethylenic bond, the residual ethylenic unsaturations after the polymerization may generally be hydrogenated. Thus, in a known manner, the polymerization of isoprene leads, after hydrogenation, to the formation of ethylene-propylene block, and the polymerization of butadiene leads, after hydrogenation, to the formation of ethylene-butylene block. Examples of these polymers include block copolymers, for example, diblock and triblock copolymers such as polystyrene/polyisoprene (SI), polystyrene/polybutadiene (SB), such as those sold under the name LUVITOL HSB by BASF, polystyrene/copoly(ethylene-propylene) (SEP), such as those sold under the name KRATON by Shell Chemical Co., and polystyrene/copoly(ethylene-butylene) (SEB). For example, KRATON G1650 (SEBS), KRATON G1651 (SEBS), KRATON G1652 (SEBS), KRATON G1657X (SEBS), KRATON G1701X (SEP), KRATON G1702X (SEP), KRATON G1726X (SEB), KRATON D-1101 (SBS), KRATON D-1102 (SBS) or KRATON D-1107 (SIS) can be used. The polymers are generally referred to as copolymers of hydrogenated or non-hydrogenated dienes.

GELLED PERMETHYL 99A-750, 99A-753-59 and 99A-753-58 (mixture of triblock polymer and star polymer), VERSAGEL 5960 from Penreco (triblock polymer+star polymer), and OS129880, OS129881 and OS84383 from Lubrizol (styrene/methacrylate copolymer) can also be used.

Examples of block or graft block copolymers comprising at least one block resulting from the polymerization of at least one ethylenic monomer comprising at least one ethylenic bond and at least one block of an acrylic polymer, include poly(methyl methacrylate)/polyisobutylene diblock and triblock copolymers and graft copolymers comprising a poly(methyl methacrylate) backbone and polyisobutylene grafts.

Examples of block or graft block copolymers comprising at least one block resulting from the polymerization of at least one ethylenic monomer comprising at least one ethylenic bond and at least one block of a polyether such as a C₂-C₁₈ polyalkylene (for example, polyethylenated and/or polyoxypropylenated), include polyoxyethylene/polybutadiene and polyoxyethylene/polyisobutylene diblock and triblock copolymers.

When a random polymer is used as the at least one stabilizer, it may be chosen such that it has a sufficient amount of groups making it soluble in the intended synthesis solvent.

It is thus possible to use copolymers based on alkyl acrylates or methacrylates derived from C₁-C₄ alcohols and alkyl acrylates or methacrylates derived from C₈-C₃₀ alcohols. For example, a stearyl methacrylate/methyl methacrylate copolymer may be used.

When the polymer synthesis solvent is apolar, the at least one stabilizer may be chosen from polymers which provide the most complete coverage possible of the particles, a plurality of stabilizing polymer chains then being absorbed onto a polymer particle obtained by polymerization.

In this case, it is possible to use, for example, as the at least one stabilizer either a graft polymer or a block polymer, so as to have better interfacial activity. The reason for this is that blocks or grafts which are insoluble in the synthesis solvent provide a more voluminous coverage at the surface of the particles.

When the liquid synthesis solvent comprises silicone oil, the at least one stabilizer may, for example, be chosen from block and graft block copolymers comprising at least one block of polyorganosiloxane and at least one block chosen from blocks of a radical polymer, blocks of a polyether, and blocks of a polyester such as polyoxypropylenated and/or oxyethylenated blocks.

When the liquid fatty phase comprises no silicone oil, the at least one stabilizer may, for example, be chosen from the following:

(a) block and graft block copolymers comprising at least one block of polyorganosiloxane and at least one block of a radical polymer or of a polyether or a polyester;

(b) copolymers of alkyl acrylates and methacrylates derived from C₁-C₄ alcohols, and of alkyl acrylates or methacrylates derived from C₈-C₃₀ alcohols; and

(c) block and graft block copolymers comprising at least one block resulting from the polymerization of at least one ethylenic monomer comprising conjugated ethylenic bonds, and at least one block chosen from blocks of vinyl and acrylic polymers, blocks of polyethers, and blocks of polyesters.

For example, diblock polymers may be used as the at least one stabilizer.

The polymers in dispersion which can be used in the first composition may have a glass transition temperature (T_{g}) ranging from −100° C. to 300° C., for example, from −50° C. to 100° C., and, even further, for example, from −10° C. to 50° C.

The at least one plasticizer for the particular polymer can be added to the polymer particles in dispersion so as to lower the T_{g} of the polymer film and to increase the adhesion of the polymer film to its substrate, for example, a keratin material. The at least one plasticizer lowers, for example, the glass transition temperature of the polymer by at least 1, 2, 3 or 4° C., for example, from 5° C. to 20° C. For example, the at least one plasticizer lowers the glass transition temperature of the polymer by at least 1, 2, 3 or 4° C., for example, from 5° C. to 20° C., when the at least one plasticizer represents no more than 10% by weight of the polymer.

In one embodiment, the at least one polymer plasticizer is chosen from plasticizers having a solubility parameter δₕ ranging from 5.5 to 11, further, for example, from 5.9 to 11, further, for example, from 7 to 10.5, further, for example, from 8 to 10, and, even further, for example, from 9 to 10 (J/cm³)^{1/2}.

The solubility parameter δₚ of the at least one plasticizer may, for example, range from 1.5 to 4.5 (J/cm³)^{1/2}, further, for example, from 1.5 to 4 (J/cm³)^{1/2}, further, for example, from 1.5 to 3.5 (J/cm³)^{1/2} and, even further, for example, from 2 to 3 (J/cm³)^{1/2}.

The at least one plasticizer may, for example, be liquid at ambient temperature and atmospheric pressure. It may, for example, be tributoxyethyl phosphate.

The definition of the solubility parameters according to Hansen is well known to the person skilled in the art and is described, for example, in the article by C. M. Hansen: “The Three Dimensional Solubility Parameters” J. Paint Technol. 39, 105 (1967). These parameters are also described in JP-A-08-109121 from Kao and in the document by D. W. Van Krevelen “Properties of Polymers” (1990), p. 190.

According to this Hansen space:

δₚ characterizes the Debye interaction forces between permanent dipoles; and

δₕ characterizes the specific interaction forces (of hydrogen bond, acid/base, donor/receptor type, etc.).

The parameters δₚ and δₕ are generally expressed in (J/cm³)^{1/2}. They are determined at ambient temperature (25° C.) and, for example, according to the calculation method indicated in the above Kao patent.

In the first composition, it is possible to use a mixture of plasticizers satisfying the above conditions. In this case the solubility parameters of the mixture are determined from those of the plasticizers taken separately, according to the following relationships:
δₚₘᵢₓ=Σxiδₚᵢ and δₕₘᵢₓ=δxiδₕᵢ
wherein xi represents the volume fraction of the at least one plasticizer (i) in the mixture.

It is within the scope of the person skilled in the art to determine the amount the at least one plasticizer so as to obtain a mixture of plasticizers meeting the above conditions.

In this embodiment, the at least one plasticizer may be chosen from esters of at least one carboxylic acid comprising from 1 to 7 carbon atoms and a polyol comprising at least 4 hydroxyl groups.

The polyol may be chosen, for example, from monosaccharide, polyhydroxyaldehyde (aldose) and polyhydroxyketone (ketose)—which is optionally cyclized. The polyol may, for example, be a monosaccharide cyclized in hemiacetal form.

The polyol may also be a polyol derived from a monosaccharide, such as erythritol, xylitol or sorbitol.

Examples of aldoses include D-ribose, D-xylose, L-arabinose, D-glucose (or alpha-D-glucopyranose when in cyclic hemiacetal form), D-mannose and D-galactose.

Examples of ketoses include D-xylulose and D-fructose (or beta-D-fructofuranose when in cyclic hemiacetal form).

The polyol may be a monosaccharide or a polysaccharide comprising from 1 to 10 monosaccharide units, for example, from 1 to 4, further, for example, 1 or 2 monosaccharide units. The polyol may be chosen from erythritol, xylitol, sorbitol, glucose and sucrose.

The polyol may, for example, be a disaccharide. Examples of disaccharides that can be used include sucrose (alpha-D-glucopyranosyl-(1-2)-beta-D-fructofuranose), lactose (beta-D-galactopyranosyl-(1-4)-beta-D-glucopyranose) and maltose (alpha-D-glucopyranosyl-(1-4)beta-D-glucopyranose).

The polyol may be a polysaccharide comprising at least two identical monosaccharide units or at least two different monosaccharide units. The ester may comprise a polyol substituted with at least two different monocarboxylic acids or with at least three different monocarboxylic acids.

The ester may be obtained by polymerizing two esters, for example, by polymerizing i) a sucrose substituted by benzoyl groups and ii) a sucrose substituted by at least one group chosen from acetyl and isobutyryl groups.

The acid may, for example, be a monocarboxylic acid chosen, for example, from acids comprising 1 to 7 carbon atoms, for example, from 1 to 5 carbon atoms, for example, acetic, n-propanoic, isopropanoic, n-butanoic, isobutanoic, tert-butanoic, n-pentanoic and benzoic acids.

The ester may be obtained from at least two different monocarboxylic acids.

In one embodiment, the acid may be an unsubstituted linear or branched acid.

The acid may, for example, be chosen from acetic acid, isobutyric acid and benzoic acid.

In embodiment, the ester may be a sucrose diacetate hexa(2-methylpropanoate).

In another embodiment, the at least one plasticizer may be chosen from esters of aliphatic and aromatic polycarboxylic acids and aliphatic and aromatic alcohols comprising from 1 to 10 carbon atoms.

The aliphatic and aromatic alcohols comprising from 1 to 10 carbon atoms, for example, from 1 to 8, and further, for example, from 1 to 6 carbon atoms. The aliphatic and aromatic alcohols may be chosen from alcohols R₁OH, wherein R₁ is chosen from methyl, ethyl, propyl, isopropyl, butyl, hexyl, ethylhexyl, decyl, isodecyl, and benzyl substituted with at least one alkyl comprising from 1 to 6 carbon atoms.

The aliphatic and aromatic polycarboxylic acids may comprise, for example, from 3 to 12 carbon atoms, further, for example, from 3 to 10 carbon atoms, further, for example, from 3 to 8 carbon atoms, and, even further, for example, 6 or 8 carbon atoms.

The aliphatic and aromatic polycarboxylic acids may, for example, be chosen from dicarboxylic acids and tricarboxylic acids.

Examples of dicarboxylic acids include those corresponding to the formula HOOC—(CH₂)ₙ—COOH wherein n is a number ranging from 1 and 10, for example, from 2 to 8, and, further, for example 2, 4, 6 or 8.

For example, dicarboxylic acids chosen from succinic, adipic and sebacic acids may be used.

The dicarboxylic acids may also be chosen from phthalic acid and the derivatives thereof, for example, butyl benzyl phthalate, dibutyl phthalate, diethylhexyl phthalate, diethyl phthalate and dimethyl phthalate.

Examples of tricarboxylic acids include the triacids corresponding to the formula 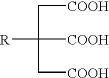
wherein: R is a hydrocarbon radical, for example, chosen from H, —OH and —OCOR′ wherein R′ is chosen from alkyls comprising, for example, from 1 to 6 carbon atoms.

The tricarboxylic acids are chosen, for example, from acetylcitric acid and derivatives thereof.

Examples of citrates include tributyl acetylcitrate, triethyl acetylcitrate, triethylhexyl acetylcitrate, trihexyl acetylcitrate, trihexyl butyroylcitrate, isodecyl citrate, isopropyl citrate, tributyl citrate and triethylhexyl citrate. In one embodiment, the carboxylic acid is not tributyl acetylcitrate.

Examples of adipates include dibutyl adipate and di-2-ethylhexyl adipate. In one embodiment, the at least one plasticizer is not diisopropyl adipate.

Examples of sebacates include dibutyl sebacate, diethylhexyl sebacate, diethyl sebacate and diisopropyl sebacate.

Examples of succinates include diethylhexyl succinate and diethyl succinate.

The at least one plasticizer may, for example, be chosen from compounds having a molecular mass of less than 5,000 g/mol, for example, less than 2,000 g/mol, further, for example, less than 1,000 g/mol, for example, less than 900 g/mol. The molecular mass of the at least one plasticizer may, for example, be greater than 100 g/mol.

The at least one plasticizer may, for example, comprise no polar group, for example, no hydroxyl group. The polar groups may, for example, be chosen from ionic and non-ionic polar groups chosen from —COOH; —OH; ethylene oxide; propylene oxide; —PO₄; —NHR; —NR₁R₂, wherein R₁ and R₂, which may be identical or different, are each chosen from linear and branched C₁ to C₂₀ alkyl and alkoxy radicals or, R₁ and R₂ may optionally form at least one ring.

The at least one plasticizer may, for example, be present in an amount ranging from 0.1 to 25% by weight, further, for example, from 0.5 to 15% by weight, and, even further, for example, from 3 to 15% by weight, relative to the total weight of the first composition.

The mass ratio between the polymer particles and the at least one plasticizer may, for example, range from 0.5:1 to 100:1, further, for example, from 1:1 to 50:1, further, for example, from 1:1 and 10:1, and, even further, for example, from 1:1 to 5:1.

Fatty Phase of the First Composition

The liquid fatty phase of the first composition may comprise any cosmetically or dermatologically acceptable, and physiologically acceptable, oil, chosen, for example, from at least one of oils of mineral, vegetable and synthetic origin, including carbon-based oils, hydrocarbon oils, fluoro oils and silicone oils, insofar as they form a homogeneous and macroscopically stable mixture and they are compatible with the intended use.

The total liquid fatty phase of the composition may be present in an amount ranging from 5 to 90% by weight, relative to the total weight of the first composition and, further, for example, from 20 to 85% by weight, relative to the total weight of the first composition. For example, the total liquid fatty phase may be at least 30%, by weight of the total weight of the first composition. For example, the fatty phase may include at least one volatile oil.

Volatile Oils of the Fatty Phase

The fatty phase, of the first composition, may further comprise at least one volatile oil.

The at least one volatile oil may be chosen, for example, from hydrocarbon oils and silicone oils optionally comprising at least one group chosen from alkyl and alkoxy groups, pendantly or at the end of the silicone chain.

The volatile silicone oils which can be used may be chosen, for example, from linear and cyclic silicones having a viscosity at ambient temperature of less than 8 cSt and comprising, for example, from 2 to 7 silicon atoms, these silicones optionally comprising at least one group chosen from alkyl and alkoxy groups comprising from 1 to 10 carbon atoms. Examples of volatile silicone oils which can be used include octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane and mixtures thereof.

The at least one volatile oil may also, for example, be chosen from C₈-C₁₆ isoalkane oils (also called isoparaffins) such as isododecane, isodecane, isohexadecane and, for example, the oils sold under the trade names ISOPAR and PERMETHYL, and, for example, isododecane (Permethyl 99A).

The at least one volatile oil may be present in an amount ranging from 5 to 85% by weight, relative to the total weight of the first composition, for example, from 20 to 75% by weight, relative to the total weight of the first composition.

Synthesis Solvent of the Polymer Particles

The polymer dispersion may be prepared as described in EP-A-749 747.

A mixture is prepared comprising the initial monomers and a free-radical initiator. This mixture is dissolved in a solvent which for the remainder of the present description is referred to as the “synthesis solvent”. When the fatty phase is a non-volatile oil, the polymerization may be carried out in an apolar organic solvent (synthesis solvent) and then the non-volatile oil (which must be miscible with the said synthesis solvent) is added and the synthesis solvent distilled off selectively.

A synthesis solvent is chosen such that the initial monomers and the free-radical initiator are soluble therein and the polymer particles obtained are insoluble therein, so that they precipitate from the solvent as they are formed. The synthesis solvent may, for example, be chosen from alkanes such as heptane, isododecane and cyclohexane.

When the fatty phase chosen is the at least one volatile oil the polymerization may be carried out directly in the oil, which hence also acts as the synthesis solvent. The monomers and the free-radical initiator must also be soluble therein, and the polymer obtained must be insoluble therein.

The monomers may, for example, be present in the synthesis solvent, prior to polymerization, at 5-20% by weight of the reaction mixture. The total amount of the monomers may be present in the solvent before the beginning of the reaction, or a fraction of the monomers may be added at the rate at which the polymerization reaction proceeds.

The free-radical initiator may, for example, be azobisisobutyronitrile or tert-butyl peroxy-2-ethylhexanoate.

The volatile phase of the composition may comprise the synthesis solvent of the dispersed polymer particles.

Non-Volatile Oil of the Fatty Phase

The fatty phase may, for example, comprise at least one apolar or relatively non-polar non-volatile oil.

Examples of relatively non-polar non-volatile oils which can be used include apolar oils and, for example, oils comprising at least one alkyl chain, for example, at least one alkyl chain chosen from C₃-C₄₀ alkyl chains. Further examples of apolar and relatively non-polar oils include at least one of the following:

linear and branched hydrocarbons such as liquid paraffin, vaseline oil and light naphthalene oil and hydrogenated polyisobutene;

hydrocarbon oils of animal origin such as squalene;

vegetable hydrocarbon oils such as the liquid triglycerides of fatty acids of at least 10 carbon atoms;

synthetic esters and ethers, for example, fatty acids, such as the oils of formula R₁CO(O)ₓR₂, wherein R₁ is chosen from residues of an acid comprising from 2 to 29 carbon atoms, wherein x is 0 or 1, and R₂ is chosen from hydrocarbon chains comprising from 3 to 30 carbon atoms, such as, tributyl acetylcitrate, oleyl erucate, 2-octyldodecyl behenate, triisoarachidyl citrate, isocetyl stearoylstearate and octyldodecanyl stearoylstearate, n-propyl acetate, tridecyl trimellitate, dodecane dioleate and diisocetyl stearate, arachidyl propionate, dibutyl phthalate, propylene carbonate and octyldodecyl pentanoate; polyol esters such as vitamin F, sorbitan isostearate, glyceryl triisostearate and diglycerol; silicone oils such as polydimethylsiloxanes (PDMS), optionally comprising at least one chain chosen from C₃-C₄₀ alkyl and alkoxy chains and a phenyl chain, such as phenyltrimethicones, polyalkylmethylsiloxanes, optionally fluorinated such as polymethyltrifluoropropyldimethylsiloxanes, and/or substituted with at least one functional group, for example, chosen from hydroxyl, thiol and amine groups; polysiloxanes modified with fatty acids, fatty alcohols and polyoxyalkylenes; and fluoro oils.

The at least one non-volatile apolar or relatively non-polar oils may be present in an amount ranging from 0.1 to 20% by weight, for example, from 0.5 to 10% by weight, and, even further, for example, from 1 to 5% by weight, relative to the total weight of the first composition.

The at least one non-volatile oil may, for example, be apolar. It may, for example, be chosen from hydrocarbons, for example, alkanes, such as hydrogenated polyisobutene.

The fatty phase may also comprise polar oils chosen from at least one of fatty acid esters comprising from 7 to 29 carbon atoms such as diisostearyl malate, isopropyl palmitate, diisopropyl adipate, triglycerides of caprylic/capric acids, such as those sold by Stearineries Dubois or those sold under the names MIGLYOL. 810, 812 and 818 by Dynamit Nobel, the oils of shea butter, isopropyl myristate, butyl stearate, hexyl laurate, diisopropyl adipate, isononyl isononanoate, 2-hexyldecyl laurate, 2-octyidecyl palmitate, 2-octyldodecyl myristate or lactate, di-2-ethylhexyl succinate, 2-ethylhexyl palmitate, 2-octyldodecyl stearate, castor oil; esters of lanolic acid, lauric acid and stearic acid; higher fatty alcohols (of 7 to 29 carbon atoms) such as stearyl alcohol, linoleyl alcohol and linolenyl alcohol, isostearyl alcohol, 2-octyldodecanol, decanol, dodecanol, octadecanol and oleyl alcohol; higher fatty acids (of 7 to 29 carbon atoms) such as myristic acid, palmitic acid, stearic acid, behenic acid, oleic acid, linoleic acid, linolenic acid and isostearic acid.

These non-volatile polar oils may be present in an amount ranging from 0.1 to 10% by weight, relative to the total weight of the first composition and, for example, from 1 to 5% by weight, relative to the total weight of the first composition.

For example, the liquid fatty phase may comprise at least one apolar volatile oil and at least one apolar non-volatile oil.

Gelling Agent of the First Composition

In one embodiment, the first composition may comprise at least one gelling agent as described above.

Colloidal Dispersion of the First Composition

The first composition may, for example, comprise particles which are solid at ambient temperature and are dispersed in the physiologically acceptable medium, and which are introduced into the composition in the form of a colloidal dispersion, also referred to as a “particle paste”, as described in Patent Application No. WO 02/39961, the disclosure of which relates to such colloidal dispersions is specifically incorporated herein by reference.

As used herein, a “colloidal dispersion” or “particle paste” is a concentrated colloidal dispersion of coated or uncoated particles in a continuous medium, this dispersion being stabilized using a dispersant or, where appropriate, without a dispersant. These particles may be chosen from at least one of pigments, nacres, and solid fillers. These particles may be of any form, for example, spherical or elongated such as fibres. They may be insoluble in the medium.

The purpose of the dispersant is to protect the dispersed particles against agglomeration or flocculation. The concentration of dispersant that may be used, for example, to stabilize the colloidal dispersion ranges from 0.3 to 5 mg/m², and, further, for example, from 0.5 to 4 mg/m² of particle surface area. The dispersant may be a surfactant, an oligomer, a polymer or a mixture of two or more of these, and carries at least one functionality which has a high affinity for the surface of the particles to be dispersed. For example, the dispersants are able to attach physically or chemically to the surface of the pigments. These dispersants additionally exhibit at least one functional group which is compatible with or soluble in the continuous medium. The dispersants may be chosen from at least one of esters of 12-hydroxystearic acid, for example, and of C₈ to C₂₀ fatty acid and polyol, such as glycerol or diglycerol can be used, examples include poly(12-hydroxystearic) stearate with a molecular weight of approximately 750 g/mol, such as that sold under the name SOLSPERSE 21 000 by Avecia, polyglyceryl-2 dipolyhydroxystearate (CTFA name), sold under the reference DEHYMULS PGPH by Henkel, and also polyhydroxystearic acid, such as that sold under the reference ARLACEL P100 by Uniqema.

As other dispersants which can be used in the first composition, examples include quaternary ammonium derivatives of polycondensed fatty acids, such as SOLSPERSE 17 000 sold by Avecia and the polydimethylsiloxane/oxypropylene mixtures such as those sold by Dow Corning under the references DC-2-5185 and DC2-5225 C.

Polydihydroxystearic acid and the esters of 12-hydroxystearic acid may, for example, be intended for a hydrocarbon-based or fluorine-based medium, while the dimethylsiloxane oxyethylene/oxypropylene mixtures may, for example, be intended for a silicone-based medium.

The colloidal dispersion is a suspension of particles, for example, of micron size (<10 μm) in a continuous medium. The volume fraction of particles in a concentrated dispersion ranges from 20 to 40%, for example, greater than 30%, corresponding to a weight fraction which can range up to 70% depending on the density of the particles.

The particles dispersed in the medium may be chosen from at least one of mineral particles and organic particles, such as those described below.

The continuous medium of the paste may be any medium and may comprise any solvent or liquid fatty substance and mixtures thereof. For example, the liquid medium of the particle paste is one of the liquid fatty substances or oils which is desired to use in the composition, hence forming part of the liquid fatty phase.

The “particle paste” or colloidal dispersion may, for example, be a “pigment paste” comprising a colloidal dispersion of colored, coated or uncoated, particles. These colored particles are pigments, nacres or a mixture of pigments and/or nacres.

The colloidal dispersion may, for example, be present in an amount ranging from 0.5 to 60% by weight, further, for example, from 2 to 40% by weight, and, even further, for example, from 2 to 30% by weight, relative to the total weight of the first composition.

The pigments may be chosen, for example, from white and colored pigments, mineral and organic pigments, interference pigments and non-interference pigments. Examples of mineral pigments include titanium dioxide, optionally with surface treatment, zirconium oxide and cerium oxide, and zinc oxide, iron oxide (black, yellow and red) and chromium oxide, manganese violet, ultramarine blue, chromium hydrate and ferric blue. Examples of organic pigments include carbon black, organic lake pigments of barium, strontium, calcium and aluminium, including those certified by the U.S. Food and Drug Administration (FDA) (examples D&C or FD&C) and those exempt from FDA certification, such as lakes based on cochineal carmine. The pigments may be present in an amount ranging from 0.1 to 50%, by weight, for example, from 0.5 to 35%, further, for example, from 2 to 25% by weight, in terms of active substance, relative to the total weight of the composition.

The nacreous pigments may be chosen from white nacreous pigments such as titanium-covered mica and bismuth oxychloride, colored nacreous pigments such as titanium mica with iron oxides, titanium mica with, for example, ferric blue and chromium oxide, titanium mica with an organic pigment of the aforementioned type, and also nacreous pigments based on bismuth oxychloride. The nacreous pigments may be present in an amount ranging from 0 to 25% by weight, for example, from 0.1 to 15% (if they are present) (in terms of active substance) by weight, of the total weight of the first composition. Pigments having goniochromatic properties may be used.

The fillers may be chosen from mineral and organic, lamellar and spherical fillers. Examples include talc, mica, silica, kaolin, Nylon % powders (Orgasol® from Atochem), poly-β-alanine powders and polyethylene powders, powders of polymers of tetrafluoroethylene (Teflon®), lauroyllysine, starch, boron nitride, hollow microspheres such as Expancel® (Nobel Industries), Polytrap® (Dow Corning) and silicone resin microbeads (Tospearls® from Toshiba, for example), precipitated calcium carbonate, magnesium carbonate and bicarbonate, hydroxyapatite, hollow silica microspheres (Silica Beads® from Maprecos), glass and ceramic microcapsules, metal soaps derived from organic carboxylic acids comprising from 8 to 22 carbon atoms, for example, from 12 to 18 carbon atoms, for example, zinc stearate, magnesium stearate and lithium stearate, zinc laurate and magnesium myristate.

Wax of the First Composition

Depending on the type of application envisaged, the first composition may further comprise at least one wax.

The at least one wax may be chosen from lipophilic fatty compounds which are solid at ambient temperature (25° C.) and exhibits a reversible solid/liquid state change, the compound having a melting temperature of greater than 40° C. and possibly up to 200° C. and having an anisotropic crystalline organization in the solid state. The size of the crystals is such that they diffract and/or scatter light, thereby endowing the composition with a turbid, more or less opaque appearance. By bringing the at least one wax to its melting temperature it can be made miscible with the oils and made to form a microscopically homogeneous mixture; however, by bringing the temperature of the mixture back to ambient temperature, the at least one wax is recrystallized in the oils of the mixture. It is this recrystallization in the mixture which is responsible for the decrease in the gloss of the mixture.

For example, linear hydrocarbon waxes may be used. Their melting point may, for example, be greater than 35° C., further, for example, greater than 55° C., and, even further, for example, greater than 80° C.

The linear hydrocarbon waxes may, for example, be chosen from substituted linear alkanes, unsubstituted linear alkanes, unsubstituted linear alkenes, and substituted linear alkenes, and unsubstituted compounds comprising solely carbon and hydrogen and the substituents, of the substituted linear alkanes and alkenes, do not comprise any carbon atoms.

Linear hydrocarbon waxes include polymers and copolymers of ethylene with a molecular weight ranging from 400 to 800, for example, POLYWAX 500 and POLYWAX 400 sold by New Phase Technologies.

Linear hydrocarbon waxes include linear paraffin waxes, such as the paraffins S&P 206, S&P 173 and S&P 434 from Strahl & Pitsch.

Linear hydrocarbon waxes include long-chain linear alcohols, such as the products comprising a mixture of polyethylene and alcohols comprising from 20 to 50 carbon atoms, for example, PERFORMACOL 425 and PERFORMACOL 550 (mixture in 20/80 proportions) sold by New Phase Technologies.

The at least one wax may be present in an amount ranging from 2 to 30% by weight, for example, from 5 to 20% by weight, and, further, for example, from 5 to 15% by weight, relative to the total weight of the first composition, so as not excessively to diminish the gloss of the first composition and of the film deposited on the lips and/or skin.

The embodiments disclosed herein are illustrated in more detail in the following examples. The percentages are percentages by weight.

### EXAMPLE 1

### Polymer Dispersion

A dispersion was prepared of an uncrosslinked copolymer of methyl acrylate and acrylic acid in a 95/5 ratio in isododecane by the method of Example 1 of EP-A-749 746, replacing the heptane by isododecane. This gave a dispersion of poly(methyl acrylate/acrylic acid) particles surface-stabilized in isododecane by a block polystyrene/copoly(ethylene-propylene) diblock copolymer sold under the name KRATON G1701, having a solids content of 25% by weight.

### EXAMPLE 2

### Lipstick

| | |
|---|---|
| Polyethylene wax | 10.5% |
| (weight-average molecular mass: 500) | |
| Linear fatty alcohols | +111 2.5% |
| (Performacol 550 alcohol, sold by | |
| New Phase Technologies) | |
| Dispersion of Example 1 | 68% |
| Sucrose acetate isobutyrate | 5% |
| (Eastman SAIB sold by Eastman Chemical) | |
| Pigment paste | 13.5% |
| Fragrance | 0.5% |

A heating vessel was charged with the polyethylene wax, the C30-C50 alcohols and the pigment paste, which were heated at 100° C. with magnetic stirring to give a homogeneous mixture. The composition of the pigment paste was 70% pigments, 1% poly(12-hydroxystearic) stearate and 29% hydrogenated polyisobutene. Subsequently the dispersion of Example 1 and the sucrose acetate isobutyrate were added, while maintaining the temperature and the stirring until the mixture homogenized. The composition was cast into molds. The resulting sticks were homogeneous in color and went on well. They resulted in a deposit on the lips which had good staying power, did not migrate or transfer and was not sticky.

### EXAMPLE 3

### Lipstick as First Composition

Polystyrene/poly(ethylene-propylene)

| | |
|---|---|
| Polystyrene/poly(ethylene-propylene) | 0.5% |
| Kraton G-1650E | |
| (sold by Kraton) | |
| Dispersion of Example 1 | 65% |
| Polycaprolactone | 10% |
| (Capa 2125, manufactured by Solvay) | |
| Sucrose acetate isobutyrate | 10% |
| (Eastman SAIB, sold by Eastman Chemical) | |
| Pigmeent paste | 13.5% |
| Fragrance | qs |
| Preservative | qs |

The polycaprolactone was melted at 10° C. and then the pigment paste, whose composition was identical to that of Example 2, was added with stirring. Subsequently, the other ingredients were added until a homogeneous mixture was obtained. The mixture was subsequently cooled to ambient temperature and packaged in pots.

### EXAMPLE 4

### Second Composition

| | |
|---|---|
| Polydimethylsiloxane sold | 40% |
| as Silbiomne 70047 V by Rhodia | |
| (500 000 cSt-250 000 g/mol) | |
| Polydimethylsiloxane sold | 60% |
| by Dow Corning as DC200 (5 cSt) | |

The two ingredients were mixed at 70° C. using a Rayneri stirrer.

The second composition had a viscosity of approximately 8±1 Pa·s, the viscosity being measured at 25° C. with a Rheomat RM 180 instrument, using spindle 4 rotating at 200 rpm, after 1.0 minutes in accordance with measurement system 75.

1. A two-coat cosmetic makeup product comprising at least two distinct compositions wherein,
a first composition comprises a physiologically acceptable medium and
a second composition comprises a mixture of at least one high molecular weight polymer having a weight-average molecular mass of greater than or equal to 200,000 g/mol and at least one low molecular weight non-volatile liquid silicone compound having a weight-average molecular mass of less than 200,000 g/mol, wherein the at least one high molecular weight polymer and the at least one low molecular weight non-volatile liquid silicone are present in a proportion such that the dynamic viscosity of the mixture at 25° C., measured with a Mettler RM 180 rotational viscometer, ranges from 0.1 Pa·s to 120 Pa·s.

2. A two-coat cosmetic makeup product comprising at least two distinct compositions wherein,
a first composition comprises a physiologically acceptable medium and
a second composition comprises a mixture of at least one high molecular weight polymer having a weight-average molecular mass of greater than or equal to 200,000 g/mol and at least one low molecular weight non-volatile liquid silicone compound having a weight-average molecular mass of less than 200,000 g/mol, wherein the at least one high molecular weight polymer and the at least one low molecular weight non-volatile liquid silicone are present in a proportion such that the dynamic viscosity of the mixture at 25° C., measured with a Mettler RM 180 rotational viscometer, ranges from 0.1 Pa·s to 120 Pa·s, and
wherein the second composition is applied on top of the first composition.

3. The cosmetic makeup product according to claim 1, wherein the at least one high molecular weight polymer is chosen from silicone polymers.

4. The cosmetic makeup product according to claim 1, wherein the dynamic viscosity of the mixture ranges from 0.5 Pa·s to 50 Pa·s.

5. The cosmetic makeup product according to claim 4, wherein the dynamic viscosity of the mixture ranges from 3.5 Pa·s to 25 Pa·s.

6. The cosmetic makeup product according to claim 5, wherein the dynamic viscosity of the mixture ranges from 5 Pa·s to 10 Pa·s.

7. The cosmetic makeup product according to claim 1, wherein the at least one high molecular weight polymer has a weight-average molecular mass ranging from 200,000 g/mol to 4,000,000 g/mol.

8. The cosmetic makeup product according to claim 7, wherein the at least one high molecular weight polymer has a weight-average molecular mass ranging from 200,000 g/mol to 2,500,000 g/mol.

9. The cosmetic makeup product according to claim 8, wherein the at least one high molecular weight polymer has a weight-average molecular mass ranging from 200,000 g/mol to 2,000,000 g/mol.

10. The cosmetic makeup product according to claim 1, wherein the at least one high molecular weight polymer is chosen from silicone polymers of formula (A): 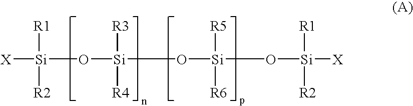
wherein:
R₁, R₂, R₅ and R₆, which may be identical or different, are each chosen from alkyl radicals comprising from 1 to 6 carbon atoms, wherein the carbon atoms are optionally substituted by at least one fluorine atom;
R₃ and R₄, which may be identical or different, are each chosen from alkyl radicals comprising from 1 to 6 carbon atoms and an aryl radical;
X, which may be identical or different, is chosen from alkyl radicals comprising from 1 to 6 carbon atoms, a hydroxyl radical, a vinyl radical, an allyl radical and alkoxy radicals comprising from 1 to 6 carbon atoms; and
n and p, which may be identical or different, are chosen such that the at least one high molecular weight polymer has a weight-average molecular mass of greater than or equal to 200,000 g/mol.

11. The cosmetic makeup product according to claim 10, wherein the at least one high molecular weight silicone polymer is a dimethicone of formula (A) wherein R₁ to R₆ and X are methyls.

12. The cosmetic makeup product according to claim 11, wherein the at least one high molecular weight silicone polymer is a dimethicone of formula (A) wherein the value of n is chosen such that the weight-average molecular mass of the at least one high molecular weight compound ranges from 200,000 g/mol to 600,000 g/mol.

13. The cosmetic makeup product according to claim 12, wherein the value of n is chosen such that the weight-average molecular mass of the at least one high molecular weight silicone polymer ranges from 200,000 g/mol to 300,000 g/mol.

14. The cosmetic makeup product according to claim 13, wherein the value of n is chosen such that the weight-average molecular mass of the at least one high molecular weight silicone polymer ranges from 240,000 g/mol to 260,000 g/mol.

15. The cosmetic makeup product according to claim 10, wherein the at least one high molecular weight silicone polymer is a dimethiconol of formula (A), wherein R₁ to R₆ are each a methyl group, X is a hydroxyl group, p=0, and n is a number ranging from 2,000 to 40,000.

16. The cosmetic makeup product according to claim 15, wherein n is a number ranging from 3,000 to 30,000.

17. The cosmetic makeup product according to claim 1, wherein the at least one high molecular weight polymer has a viscosity at 25° C. ranging from 10,000 cSt to 10,000,000 cSt, measured in accordance with standard ASTM D-445.

18. The cosmetic makeup product according to claim 17, wherein the at least one high molecular weight polymer has a viscosity at 25° C. ranging from 10,000 cSt to 5,000,000 cSt, measured in accordance with standard ASTM D-445.

19. The cosmetic makeup product according to claim 18, wherein the at least one high molecular weight polymer has a viscosity at 25° C. ranging from 10,000 cSt to 1,000,000 cSt, measured in accordance with standard ASTM D-445.

20. The cosmetic makeup product according to claim 19, wherein the at least one high molecular weight polymer has a viscosity at 25° C. ranging from 100,000 cSt to 1,000,000 cSt, measured in accordance with standard ASTM D445.

21. The cosmetic makeup product according to claim 20, wherein the at least one high molecular weight polymer has a viscosity at 25° C. ranging from 300,000 cSt to 700,000 cSt, measured in accordance with standard ASTM D-445.

22. The cosmetic makeup product according to claim 1, wherein the second composition is transparent.

23. The cosmetic makeup product according to claim 1, wherein the viscosity of the at least one low molecular weight non-volatile liquid silicone compound ranges from 0.5 cSt to 10,000 cSt, measured in accordance with standard ASTM D-445.

24. The cosmetic makeup product according to claim 23, wherein the viscosity of the at least one low molecular weight non-volatile liquid silicone compound ranges from 0.5 cSt to 500 cSt, measured in accordance with standard ASTM D-445.

25. The cosmetic makeup product according to claim 24, wherein the viscosity of the at least one low molecular weight non-volatile liquid silicone compound ranges from 1 cSt to 10 cSt, measured in accordance with standard ASTM D-445.

26. The cosmetic makeup product according to claim 1, wherein the weight-average molecular mass of the at least one low molecular weight non-volatile liquid silicone compound ranges from 400 g/mol to 200,000 g/mol.

27. The cosmetic makeup product according to claim 26, wherein the weight-average molecular mass of the at least one low molecular weight non-volatile liquid silicone compound ranges from 4,000 g/mol to 20,000 g/mol.

28. The cosmetic makeup product according to claim 27, wherein the weight-average molecular mass of the at least one low molecular weight non-volatile liquid silicone compound ranges from 400 g/mol to 2,000 g/mol.

29. The cosmetic makeup product according to claim 28, wherein the weight-average molecular mass of the at least one low molecular weight non-volatile liquid silicone compound ranges from 400 g/mol to 1,000 g/mol.

30. The cosmetic makeup product according to claim 1, wherein the at least one low molecular weight non-volatile liquid silicone compound is chosen from polyalkylsiloxanes, polyarylsiloxanes, and polyalkylarylsiloxanes.

31. The cosmetic makeup product according to claim 1, wherein the at least one low molecular weight non-volatile liquid silicone compound is chosen from:
non-volatile linear and branched polydimethylsiloxanes (PDMS);
polydimethylsiloxanes comprising at least one group chosen from alkyl, alkoxy and phenyl groups, pendantly or at the silicone chain end, wherein the at least one group comprises from 2 to 24 carbon atoms; and
phenylsilicones.

32. The cosmetic makeup product according to claim 31, wherein the phenylsilicones are chosen from phenyltrimethicones, phenyldimethicones, phenyltrimethylsiloxydiphenylsiloxanes, diphenyldimethicones, diphenylmethyl-diphenyltrisiloxanes and 2-phenylethyl trimethylsiloxysilicates.

33. The cosmetic makeup product according to claim 1, wherein the at least one low molecular weight non-volatile liquid silicone compound is a polydimethylsiloxane with a viscosity at 25° C., measured in accordance with standard ASTM D-445, ranging from 1 cSt to 10 cSt and/or with a molecular weight ranging from 400 g/mol to 1,000 g/mol.

34. The cosmetic makeup product according to claim 1, wherein the second composition comprises a mixture of a polydimethylsiloxane having a weight-average molecular mass of greater than or equal to 200,000 g/mol and a polydimethylsiloxane having a weight-average molecular mass of less than 200,000 g/mol.

35. The cosmetic makeup product according to claim 1, wherein the second composition comprises a mixture of a polydimethylsiloxane having a molecular mass ranging from 200,000 g/mol to 300,000 g/mol and a polydimethylsiloxane having a molecular mass ranging from 400 g/mol to 1,000 g/mol.

36. The cosmetic makeup product according to claim 1, wherein the proportion by mass of the at least one high molecular weight polymer/at least one low molecular weight non-volatile liquid silicone compound ranges from 20/80 to 60/40.

37. The cosmetic makeup product according to claim 36, wherein the proportion by mass of the at least one high molecular weight polymer/at least one low molecular weight non-volatile liquid silicone compound ranges from 35/65 to 45/55.

38. The cosmetic makeup product according to claim 1, wherein the at least one high molecular weight polymer and the at least one low molecular weight non-volatile liquid silicone compound are present in a combined amount equal to more than 70% by weight, relative to the total weight of the second composition.

39. The cosmetic makeup product according to claim 38, wherein the at least one high molecular weight polymer and the at least one low molecular weight non-volatile liquid silicone compound are present in a combined amount equal to more than 80% by weight, relative to the total weight of the second composition.

40. The cosmetic makeup product according to claim 39, wherein the at least one high molecular weight polymer and the at least one low molecular weight non-volatile liquid silicone compound are present in a combined amount equal to more than 90% by weight, relative to the total weight of the second composition.

41. The cosmetic makeup product according to claim 40, wherein the at least one high molecular weight polymer and the at least one low molecular weight non-volatile liquid silicone compound are present in a combined amount equal to more than more than 95% by weight, relative to the total weight of the second composition.

42. The cosmetic makeup product according to claim 1, wherein the second composition further comprises at least one colorant chosen from nacres, interference pigments and metallic effect pigments.

43. The cosmetic makeup product according to claim 42, wherein the second composition does not comprise wax.

44. The cosmetic makeup product according to claim 43, wherein the first composition comprises polymer particles dispersed in a liquid fatty phase.

45. The cosmetic makeup product according to claim 44, wherein the polymer is a film-forming polymer.

46. The cosmetic makeup product according to claim 45, wherein the polymer particles are chosen from at least one of polyurethanes, polyurethane-acrylics, polyureas, polyurea-polyurethanes, polyester-polyurethanes, polyether-polyurethanes, polyesters, polyester amides, fatty-chain polyesters, alkyds; acrylic polymers and copolymers, vinyl polymers and copolymers; acrylic-silicone copolymers; polyacrylamides; silicone polymers, and fluoro polymers.

47. The cosmetic makeup product according to claim 44, wherein the polymer particles are present, in terms of dry matter, in an amount ranging from 2% to 40% by weight, relative to the total weight of the first composition.

48. The cosmetic makeup product according to claim 47, wherein the polymer particles are present, in terms of dry matter, in an amount ranging from 5% to 40% by weight, relative to the total weight of the first composition.

49. The cosmetic makeup product according to claim 48, wherein the polymer particles are present, in terms of dry matter, in an amount ranging from 5% to 35% by weight, relative to the total weight of the first composition.

50. The cosmetic makeup product according to claim 49, wherein the polymer particles are present, in terms of dry matter, in an amount ranging from 8% to 30% by weight, relative to the total weight of the first composition.

51. The cosmetic makeup product according to claim 44, wherein the polymer particles of the first composition are surface-stabilized by at least one stabilizer.

52. The cosmetic makeup product according to claim 51, wherein the at least one stabilizer is chosen from block polymers and graft block polymers, wherein the block polymers and/or graft block polymers comprise at least one block resulting from the polymerization of a diene and at least one block of a vinyl polymer.

53. The cosmetic makeup product according to claim 52, wherein the at least one stabilizer is chosen from diblock polymers.

54. The cosmetic makeup product according to claim 1, wherein the first composition is in anhydrous form.

55. The cosmetic makeup product according to claim 1, wherein the cosmetic makeup product is provided in a form chosen from foundations, blushes, eyeshadows, lipsticks, care bases and care balms for the lips, eyeliners, concealers, and body makeup products.

56. The cosmetic makeup product according to claim 55, wherein the cosmetic makeup product is in the form of a lipstick.

57. The cosmetic makeup product according to claim 1, wherein the first composition is in solid form.

58. A method of making up the skin and/or lips and/or epidermal integuments, comprising,
applying to the skin, lips and/or epidermal integuments a first coat of a first composition comprising a physiologically acceptable medium and
applying over all or part of the first coat, a second coat of a second composition comprising a mixture of at least one high molecular weight polymer having a weight-average molecular mass of greater than or equal to 200,000 g/mol and at least one low molecular weight non-volatile liquid silicone compound having a weight-average molecular mass of less than 200,000 g/mol, wherein the at least one high molecular weight polymer and the at least one low molecular weight non-volatile liquid silicone are present in a proportion such that the dynamic viscosity of the mixture at 25° C., measured with a Mettler RM 180 rotational viscometer, ranges from 0.1 Pa·s to 120 Pa·s.

59. A makeup kit comprising a cosmetic makeup product comprising at least two compositions wherein,
a first composition comprises a physiologically acceptable medium and
a second composition comprises a mixture of at least one high molecular weight polymer having a weight-average molecular mass of greater than or equal to 200,000 g/mol and at least one low molecular weight non-volatile liquid silicone compound having a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa·s

60. The makeup kit according to claim 59, wherein the first and second compositions are packaged in two discrete compartments of a single container.

61. The makeup kit according to claim 60, wherein the first and second compositions are packaged in two separate containers.

62. A method for providing skin and/or lips and/or epidermal integuments with a makeup which is comfortable, glossy, does not transfer and/or does not migrate and/or has good staying power comprising including in a cosmetic makeup product comprising a first composition comprising a physiologically acceptable medium, a second composition comprising a mixture of at least one high molecular weight polymer having a weight-average molecular mass of greater than or equal to 200,000 g/mol and at least one low molecular weight non-volatile liquid silicone compound having a weight-average molecular mass of less than 200,000 g/mol, wherein the at least one high molecular weight polymer and the at least one low molecular weight non-volatile liquid silicone compound are present in a proportion such that the dynamic viscosity of the mixture at 25° C., measured with a Mettler RM 180 rotational viscometer, ranges from 0.1 Pa·s to 120 Pa.
